(19) United States
(12) Patent Application Publication
(10) Pub. No.: US 2025/0282055 A1
(43) Pub. Date: Sep. 11, 2025

(54) RAIL ASSEMBLY FOR TABLE-MOUNTED MANIPULATOR SYSTEM, AND RELATED DEVICES, SYSTEMS AND METHODS

(71) Applicant: INTUITIVE SURGICAL OPERATIONS, INC., Sunnyvale, CA (US)

(72) Inventors: Ryan C. Abbott, San Jose, CA (US); Rumen Deyanov, Fremont, CA (US)

(73) Assignee: INTUITIVE SURGICAL OPERATIONS, INC., Sunnyvale, CA (US)

(21) Appl. No.: 18/860,978

(22) PCT Filed: Apr. 28, 2023
(86) PCT No.: PCT/US2023/020320

§ 371 Date: Oct. 28, 2024

Related U.S. Application Data
(60) Provisional application No. 63/336,773, filed on Apr. 29, 2022.

(51) Int. Cl. B25J 9/16; A61B 34/35; B25J 5/02; B25J 9/00

(52) U.S. Cl. CPC B25J 9/1689; A61B 34/35; B25J 5/02; B25J 9/0084

(57) ABSTRACT

A teleoperable manipulator system comprises a table assembly, a rail coupled to the table assembly, and one or more manipulators coupled to the rail. The table assembly comprises a support column and a platform assembly coupled to the support column and configured to support a body. The platform assembly comprises lateral and longitudinal dimensions defining outer lateral and longitudinal boundaries of the platform assembly. The rail is translatable relative to the support column along a longitudinal dimension of the rail. The manipulators are translatable relative to the rail along the longitudinal dimension of the rail. The rail is configured to remain within at least the outer lateral boundaries of the platform assembly throughout all motion of the rail relative to the support column.

### CROSS-REFERENCE TO RELATED APPLICATIONS

This application claims priority to U.S. Provisional Application No. 63/336,773, filed Apr. 29, 2022, which is incorporated by reference herein in its entirety.

### FIELD

Aspects of this disclosure relate generally to table mounted manipulator systems. In particular, aspects of the disclosure relate to rail assemblies for movably mounting manipulators to a table, such as a medical system table for supporting a patient. Related devices, systems, and methods also are disclosed.

### INTRODUCTION

Computer-assisted manipulator systems (“manipulator systems”), sometimes referred to as robotically assisted systems or robotic systems, can include one or more manipulators that can be operated with the assistance of an electronic controller (e.g., computer) to move and control functions of one or more instruments when coupled to the manipulators. A manipulator generally includes mechanical links connected by joints. An instrument is removably couplable to (or permanently coupled to) one of the links, typically a distal link of the plural links.

In some computer-assisted manipulator systems, the manipulators are attached to a manipulator support structure (e.g., a patient side cart) that is separate from a support structure that supports a patient or workpiece. In other manipulator systems, the manipulators are attached directly to the support structure that supports the patient or workpiece, e.g., to an operating table. This support structure that supports the patient or workpiece may be referred to herein as a “table assembly” or “table” to simplify the description. Manipulator systems in which the manipulators are mounted to the table assembly may be referred to herein as table-mounted manipulator systems.

Table-mounted manipulator systems pose certain challenges. The space around a table assembly may need to be occupied with various pieces of equipment and/or personnel during the performance of various tasks that make up a medical procedure. Moreover, the space constraints around the table can vary depending on the medical procedure being performed, with some tasks (such as transferring a patient to the table, draping manipulators, etc.) benefiting from or being facilitated by a large amount of open space around the table. In manipulator systems including movable patient-side carts, such open space around the table can be obtained by moving the patient-side cart away from the table intended to support the patient. However, in table-mounted manipulator systems, moving the manipulators out of the way when space is desired around the table poses challenges as the manipulators are either affixed to the table or at least not practical to remove from the table during a particular stage of a medical procedure. Thus, it can be challenging to avoid interference between the manipulators and the other entities in the space around the table in a table-mounted manipulator system.

By way of example, while transferring a patient from a gurney to the table, it may be desired for a long side of the table to be substantially free of obstructions so as to allow the gurney to be placed flush with the table to ease the transfer of patient from gurney to table. But when manipulators are mounted to this long side of the table, the needed open space can be difficult to achieve as the manipulators might get in the way. As another example, while preparing a patient on the table, a number of personnel and equipment may need to be close to the patient and may occupy much of the space around the table and therefore the manipulators mounted to the table can get in the way of personnel and hinder the preparations. As another example, covering the manipulators with a sterile drape can be easier when there is a substantial amount of free space around the manipulator, but when the manipulators are attached to the table assembly it can be difficult to obtain such space due to the proximity of the table.

In addition, some table assemblies can switch between multiple spatial configurations, such as by lowering or raising sections of a multi-section table, and it can be challenging to accommodate such changes in configuration of the table assembly when the manipulators are mounted to the table assembly. Another challenge with table-mounted manipulators is that the positioning of the manipulators relative to a patient can be limited due to the manipulators being attached to the table, thus making it more difficult to reach certain portions of a patient or certain desired angles during a medical procedure.

One way to avoid the manipulators becoming an obstruction or otherwise interfering with a task is to place the manipulators in a stowed state during a stage that requires more space around the table. In the stowed state, the manipulators are generally compacted (e.g., folded) and placed in a stowed location, such as under a platform of the table assembly. However, stowing of the manipulators to make sufficient space for a task may not always be feasible, as some tasks require or benefit from the manipulators being in a deployed configuration. Moreover, even when the task being performed does not require the manipulators to be in a deployed configuration, in some circumstances stowing the manipulators during the task can be undesirable.

For example, stowing the manipulators can compromise the sterility of the manipulators, which can have various undesired effects. The stowing of the manipulators can compromise the sterility of the manipulators because the stowed location (e.g., under the table) is generally not within the sterile field established around the operating table. The sterile field is a region in which any exposed surfaces of objects in the region are maintained in a sterile condition (e.g., a condition substantially free from contaminants, such as biological pathogens, dusts, oils, etc.) and non-sterile surfaces are covered by a sterile barrier. Thus, because stowing the manipulators can compromise their sterility, the manipulators would need to be sterile (e.g., covered with a sterile drape) after completion of the task for which space around the table was needed and once the manipulators have been returned from the stowed state into the sterile field.

But such sterilization (e.g., draping) of the manipulators after completion of the task can be undesirable in some circumstances. For example, when the task involves the transferring of a patient to the table and/or preparation of the patient on the table, draping the manipulators after completion of the task will cause a delay between when the patient is ready and when subsequent tasks of the overall medical procedure can be performed. Such a delay is generally undesirable, as once the patient has been prepared for the procedure (e.g., transferred to the table) it is generally desirable to proceed with and finish the overall medical procedure as quickly as is feasible. On the other hand, prior to the patient being prepared for the procedure, time is less constrained and delays are generally more acceptable.

Thus, it is generally desired to perform as many tasks as possible prior to the preparation of the patient so as to minimize the time the patient is on the table. Another reason it can be desirable to drape the manipulators prior to transferring the patient to the table it that it is generally easier to drape the manipulators when the patient is not present, as the personnel may have more room to maneuver at that time. Another reason to drape the manipulators prior to transferring the patient to the table is to avoid having non-sterile objects in the vicinity of a sterile patient, even if only temporarily (e.g., while they are in the process of being draped). Thus, even when stowing of the manipulators to free up space is possible, the stowing of the manipulators is not always be an acceptable option to free up space for a given stage of an overall procedure.

Another way to mitigate some of the challenges noted above is to configure the system to allow relative movement between the manipulators and the table assembly. However, mechanisms for providing such relative motion between the manipulator and table generally provide a range of motion that is insufficient to allow the manipulators to move fully out of the way, and those mechanisms that do provide a sufficient range of motion are generally complicated, costly, and/or impractical and tend to themselves obstruct and interfere with other equipment (e.g., due to the mechanism protruding into spaces that would otherwise be occupied by the other equipment). For example, operating tables generally have accessory rails disposed along the side surfaces of the table, with the accessory rails being configured to hold various auxiliary equipment mounted thereto, and mechanisms for providing relative motion of manipulators relative to the table often extend into spaces that would be occupied by such auxiliary equipment attached to accessory rails of the table, thus interfering with usage of such auxiliary equipment. Generally speaking, the wider the range of motion for the manipulators provided by a mechanism, the more likely it is that the mechanism will interfere with the other equipment.

Thus, it can be difficult to provide a simple, cost effective, and practical mechanism that both enables the manipulators to move relative to the operating table with a sufficient range of motion and does not obstruct or otherwise interfering with the other equipment around the table. Accordingly, a need exists for improved table mounted manipulator systems, in particular systems with improved mechanisms for mounting the manipulators to the table.

### SUMMARY

Various embodiments of the present disclosure can solve one or more of the above-mentioned problems and/or can demonstrate one or more of the above-mentioned desirable features. Other features and/or advantages may become apparent from the description that follows.

Some embodiments relate to a teleoperated manipulator system. The system can include a table assembly that includes a support column and a platform assembly coupled to the support column and configured to support a body. The platform assembly has lateral and longitudinal dimensions defining outer lateral and longitudinal boundaries of the platform assembly. The system can also include a rail coupled to the table assembly. The rail can be translatable relative to the support column along a longitudinal dimension of the rail. The system can further include one or more manipulators can be coupled to the rail and translatable relative to the rail along the longitudinal dimension of the rail. The rail can be configured to remain within at least the outer lateral boundaries of the platform assembly throughout all motion of the rail relative to the support column.

In some embodiments, the rail is configured to remain within the outer longitudinal boundaries of the platform assembly throughout all motion of the rail relative to the support column. In some embodiments, the platform assembly includes a plurality of sections movable relative to one another to change a configuration of the platform assembly between a plurality of configurations. In some embodiments, the rail is movable between a plurality of positions to allow the platform assembly to move between the plurality of configurations without interference from the rail. In some embodiment, the plurality of sections include a first end section, one or more middle sections, and a second end section consecutively positioned along the longitudinal dimension of the platform assembly, and each of the first and second end sections are independently pivotable relative to the one or more middle sections.

In some embodiments, the system further includes one or more first carriages, each first carriage movably coupling one of the manipulators to the rail, each first carriage being drivable to cause translation of the manipulator coupled thereto relative to the rail along the longitudinal dimension of the rail, and also includes one or more second carriages movably coupling the rail to the table assembly, each second carriage being drivable to cause translation of the rail relative to the platform assembly and the support column. In some embodiments, the system can include a control system communicably connected to the first and second carriages, and the control system can be configured to drive the first and second carriages to position the rail and the manipulators.

In some embodiments, the plurality of sections can include a first section and a second section positioned adjacent to and pivotable relative to the first section, wherein in a first state the second section can be declined relative to the first section and at least a portion of the second section can occupy a given region of space, wherein the rail is translatable relative to the support column between a first position and a second position, in the first position a portion of the rail occupies the given region of space, in the second position the rail does not occupy the given region of space.

In some embodiments, the system includes a control system configured to, in response to determining a transition to the first state is going to occur, cause the rail to move from the first position to the second position. In some embodiments, the system can include one or more carriages, each carriage movably coupling one of the manipulators to the rail, each carriage being drivable in translation along the longitudinal dimension of the rail so as to cause translation of the manipulator coupled thereto relative to the rail along the longitudinal dimension of the rail.

In some embodiments, the system can include a drive mechanism operably coupled to drive translation of the one or more carriages along the longitudinal dimension of the rail. The drive mechanism can include a lead screw; one or more follower nuts engaged with the lead screw and coupled with respectively corresponding ones of the one or more carriages; and one or more actuators operably coupled to the follower nuts and configured to rotate the one or more follower nuts relative to the lead screw. One or more rail mounting carriages can movably couple the rail to the platform assembly, the one or more rail mounting carriages being drivable to cause translation of the rail relative to the platform assembly and the support column. In some embodiments, the system can further include a second lead screw; one or more second follower nuts engaged with the second lead screw and coupled with respectively corresponding ones of the one or more rail mounting carriages, and one or more second actuators operably coupled to the second follower nuts to drive translation of respectively corresponding ones of the one or more rail mounting carriages relative to the rail by driving rotation of the respectively corresponding second follower nuts relative to the second lead screw. The one or more rail mounting carriages can movably couple to the fixed rail and be translatable relative to the platform assembly along the fixed rail.

In some embodiments, the drive mechanism can include one or more cables coupled with respectively corresponding ones of the one or more carriages; one or more capstans, where the one or more cables are at least partially wrapped around the one or more capstans, respectively; and one or more actuators respectively operably coupled to the one or more capstans and configured to rotate the one or more capstans pay in or pay out, based on a direction of rotation, the one or more cables relative to the one or more capstans.

In some embodiments, the one or more carriages can include a first carriage and a second carriage and the system can further comprise a rail mounting carriage movably coupling the rail to the platform assembly. In some embodiments, the one or more cables include a first cable coupled with the first carriage, a second cable coupled with the second carriage, and a third cable coupled with the rail mounting carriage, and the one or more capstans comprises a first capstan, a second capstan, and a third capstan. The first cable can be at least partially wrapped around the first capstan, the second cable can be at least partially wrapped around the second capstan.

In some embodiments, the platform assembly is tiltable relative to the support column and the rail is configured to tilt along with the platform assembly relative to the support column. In some embodiments, the platform assembly can include a plurality of sections movable relative to one another to change a configuration of the platform assembly between a plurality of configurations, where a first section of the plurality of sections is coupled to the support column and is tiltable relative to the support column, and the rail can be coupled to the first section. The outer lateral boundaries of the platform assembly can be defined by outermost edges of the platform assembly along the longitudinal side excluding the accessory rail. In some embodiments, the rail is movably coupled to the rail mounting carriage and is configured to move along the rail mounting carriage to cause translation of the rail relative to the support column along the longitudinal dimension of the rail.

In some embodiments, the system includes a drive mechanism operably coupled to drive movement of the rail along the rail mounting carriage. In some embodiments, the drive mechanism can include a lead screw; a follower nut engaged with the lead screw and coupled with the rail and the rail mounting carriage, and an actuator operably coupled to the follower nut and configured to rotate the follower nut relative to the lead screw. In some embodiment, the drive mechanism can include a cable coupled with rail mounting carriage; a capstan, the cable at least partially wrapped around the capstan; and an actuator operably coupled to drive rotation of the capstan, where rotation of the capstan pays in or pays out, based on a direction of rotation, the cable relative to the capstan.

Some embodiments relate to a rail assembly for mounting manipulators to a table assembly. In some embodiments, the rail assembly can include a rail having a longitudinal dimension; a rail mounting carriage movably engaged with the rail and configured to couple the rail to a table assembly; and first and second manipulator mounting carriages movably engaged with the rail at an opposite side of the rail as the rail mounting carriage. In some embodiments, the rail can further include a drive mechanism housed in an interior of the rail. The drive mechanism can include a first capstan assembly, a first cable operably coupled to the first capstan assembly and to the first manipulator carriage, the first cable passing through the second manipulator carriage; a second capstan assembly, a second cable operably coupled to the second capstan assembly and the second manipulator carriage, the second cable passing through the first manipulator carriage; and a third capstan assembly and a third cable operably coupled to the third capstan assembly and the rail mounting carriage. In some embodiments, the first, second, and third capstan assemblies can be arranged in a series along the longitudinal dimension of the rail.

### BRIEF DESCRIPTION OF TUE DRAWINGS

The present disclosure can be understood from the following detailed description, either alone or together with the accompanying drawings. The drawings are included to provide a further understanding of the present disclosure and are incorporated in and constitute a part of this specification. The drawings illustrate one or more embodiments of the present teachings and together with the description explain certain principles and operation. In the drawings:

FIG. 1A is a schematic side view of an embodiment of a table-mounted manipulator system in a first state.

FIG. 1B is a schematic side view of the table-mounted manipulator system of FIG. 1A in a second state.

FIG. 1C is a schematic side view of the table-mounted manipulator system of FIG. 1A in a third state.

FIG. 2 is a schematic front view of the table-mounted manipulator system of FIG. 1A.

FIG. 3 is a perspective view of another embodiment of a table-mounted manipulator system.

FIG. 4A is a perspective view of the table-mounted manipulator system of FIG. 3.

FIG. 4B is an enlarged perspective view of a portion of the table-mounted manipulator system of FIG. 4A with a rail housing made transparent.

FIG. 5A is a side view of the table-mounted manipulator system of FIG. 4A in a first state.

FIG. 5B is a side view of the table-mounted manipulator system of FIG. 4A in a second state and illustrating a third state in dashed lines.

FIG. 5C is a side view of the table-mounted manipulator system of FIG. 4A in a fourth state and illustrating a fifth state in dashed lines.

FIG. 5D is a side view of the table-mounted manipulator system of FIG. 4A in a sixth state and illustrating a seventh state in dashed lines.

FIG. 6 is a perspective view of another embodiment of a table-mounted manipulator system in a first state.

FIG. 7 is a side view of the table-mounted manipulator system of FIG. 6 in a second state.

FIG. 8A is a perspective view of a table assembly with a rail assembly in accordance with another embodiment.

FIG. 8B is a side view of a portion of the rail assembly of FIG. 8A shown in isolation.

FIGS. 9A and 9B are a perspective side views of FIG. 8B with the rail housing removed to show translation drive mechanisms of the rail assembly, with the view of FIGS. 9A and 9B taken from opposite sides.

FIG. 10 is an isolated view of an embodiment of an actuator and capstan portion of a translation drive mechanism.

### DETAILED DESCRIPTION

As noted above, there can be certain challenges arising from having manipulators attached to a table assembly in a table-mounted manipulator system, such as challenges associated with the manipulators, personnel, and/or a table obstructing or interfering with one another during various stages of a medical procedure.

To address challenges with table mounted manipulator systems, various embodiments disclosed contemplate a table-mounted manipulator system including a table assembly and a rail that is coupled to the table assembly. The rail can be translatable relative to and along a longitudinal dimension of the table assembly. The rail supports one or more manipulators, which are translatable along the rail. The translation of the rail relative to the table assembly combined with the translation of the manipulators along the rail allows for a relatively wide range of motion of the manipulators relative to the table assembly, especially when compared with the relatively narrower range of motion that would be provided if the manipulators were to translate along a similarly dimensioned but stationary rail. The wide range of motion can beneficially allow the manipulators to be moved more fully out of the way when desired, as well as allowing more flexibility in positioning instruments supported by the manipulators.

Moreover, in various embodiments disclosed herein the rail is configured to remain within outer lateral and longitudinal boundaries of a platform of the table assembly throughout all motion of the rail (i.e., an entirety of the rail is within the boundaries of the platform of the table assembly). This helps to ensure that the rail does not itself become an obstruction or interfere with other equipment, such as auxiliary equipment mounted to an accessory rail. Thus, in embodiments disclosed herein, a large range of motion is provided for the manipulators while also avoiding having the mechanism that provides the motion interfere with other equipment.

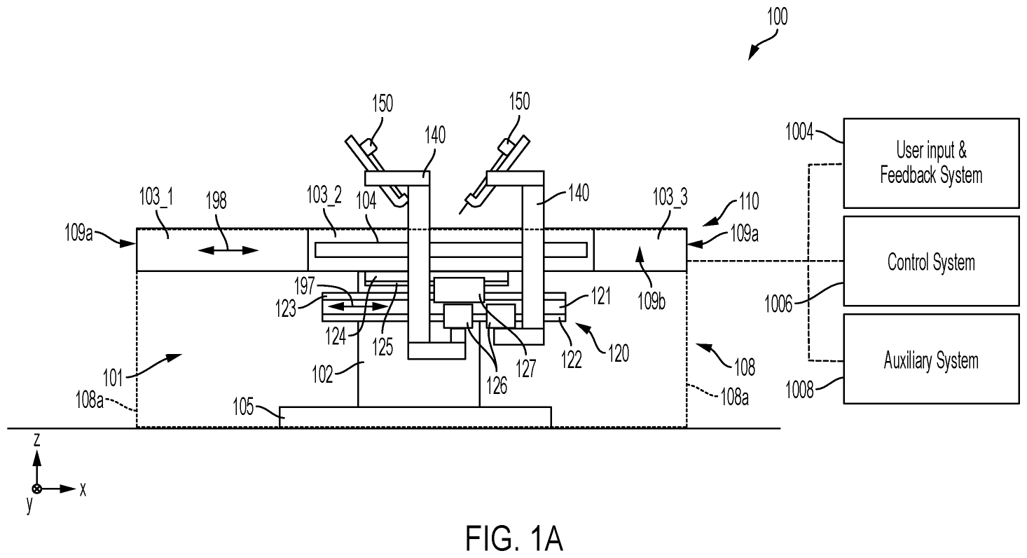

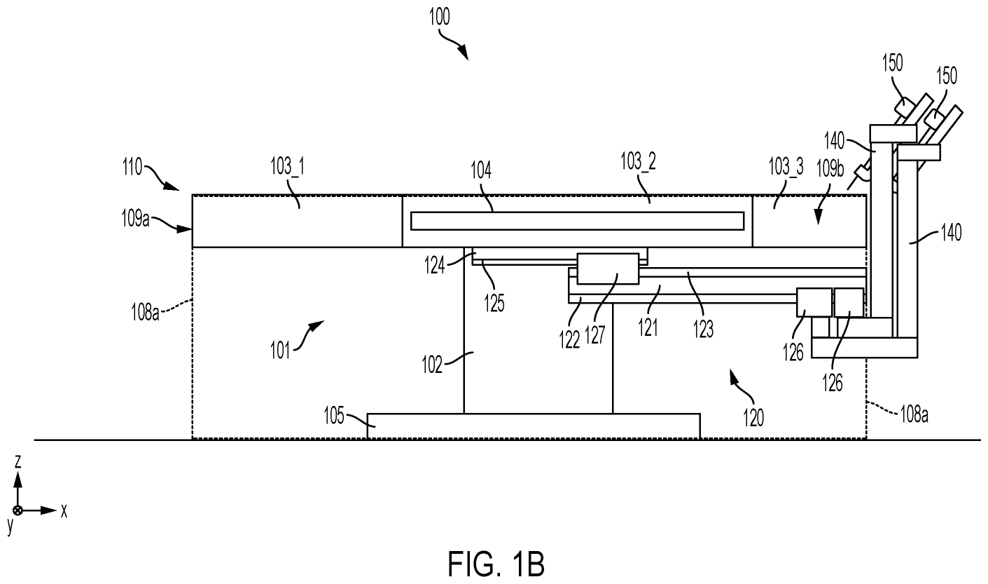

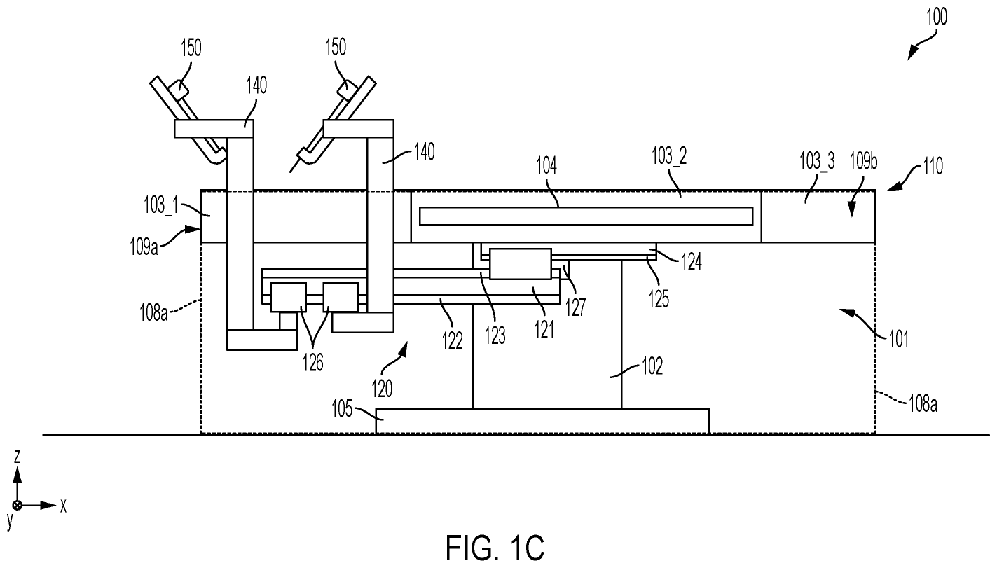

FIGS. 1A-D illustrate an embodiment of a table-mounted manipulator system 100 (“system 100”). The system 100 includes a table assembly 101, at least one rail assembly 120 coupled to the table assembly, and one or more manipulators 140 coupled to each rail assembly 120. Each manipulator 140 can carry one more instruments 150, which can be removably or permanently mounted thereon. As shown in FIG. 1A, the system 100 also can include a control system 1006, a user input and feedback system 1004, and/or an auxiliary system 1008. In some embodiments, the system 100 is configured as a computer-assisted, teleoperable medical system, in which case table assembly 101 can be configured to support a patient (not shown) and the instruments 150 are medical instruments. The system 100 in this configuration is usable, for example, to perform any of a variety of medical procedures, such as surgical procedures, diagnostic procedures, imaging procedures, therapeutic procedures, etc.

Moreover, the system 100 when configured as a teleoperable medical system need not necessarily be used on a living human patient. For example, a non-human animal, a cadaver, tissue-like materials used for training purposes, and so on, can be supported on the table assembly 101 and worked on by system 100. In other embodiments, the system 100 is configured as a computer-assisted teleoperable system for use in non-medical contexts, in which case the table assembly 101 can be configured to support an inanimate workpiece (something being manufactured, repaired, tested, etc.) and the instruments 150 can be non-medical instruments, such as industrial instruments.

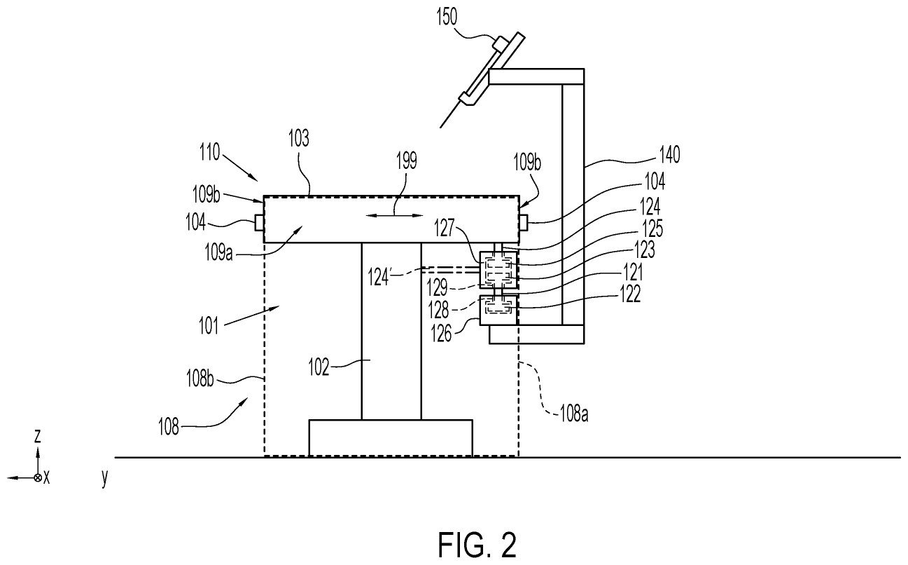

As shown in FIG. 1A, the table assembly 101 includes a platform assembly 110 configured to support the patient or inanimate workpiece, a support column 102 coupled to and supporting the platform assembly 110, and a base 105 coupled to the support column 102. The base can be configured to contact the ground or other surface upon which the table assembly 101 rests to provide stability for the table assembly 101. In some embodiments, the base 105 is omitted. In some embodiments, the base 105 includes mobility features, such as wheels, tracks, or other such features (not shown), to allow movement of the table assembly 101 along the ground or other surface. In FIGS. 1A-2, the support column 102 is illustrated as a single vertical columnar part to simplify the discussion, but the support column 102 could take any desired shape and could include any number of parts. For example, the support column 102 can include horizontal support structures (not illustrated) such as beams, rails, etc. to couple the platform assembly 110 to a vertical portion of the support column 102. Moreover, in various embodiments, the support column 102 can be telescoping and configured to extend and contract in height.

The platform assembly 110 includes one or more platform sections 103 to support the patient or workpiece. The platform sections 103 each have a support surface configured to contact and support the patient or workpiece. In some embodiments multiple platform sections 103 are used and the platform sections 103 are arranged in series to support different portions of the patient or workpiece.

For example, in the embodiment illustrated in FIG. 1A, the platform assembly 110 includes a first end section 103_1, one or more middle sections 103_2, and a second end section 103_3, with the one or more middle sections 103_2 being arranged between the two end sections 103_1 and 103_3. In some embodiments, the first end section 103_1 is configured to support a head of the patient, the second end section 103_3 is configured to support the feet and/or legs of the patient, and the one or more middle sections 103_2 are configured to support a torso and/or other portions of the patient. For convenience, the side of the platform assembly 110 that is near the first end section 103_1 (e.g., a left side in the orientation shown in FIG. 1A) will be referred to herein as a “head” of the platform assembly 110 (or “head side” or “head end”) and the side of the platform assembly 110 that is near the second end section 103_3 (e.g., a right side in the orientation shown in FIG. 1A) will be referred to herein as a “foot” of the platform assembly 110 (or “foot side” or “foot end”), but this is merely an arbitrary convention chosen herein for convenience of description and is not intended to limit the configuration or usage of the table assembly 101 (e.g., a patients head could be positioned at the “foot” side of the platform assembly 110 if desired, and vice versa). The relative positions of two components or of two portions of a single component also can be described using “head” and “foot” (e.g., a “head end” and a “foot end” of a rail 121) with “head” referring to the component or portion that is relatively closer to the head end of the platform assembly 110 and “foot” referring to the component or portion that is relative closer to the foot end of the platform assembly 101.

In other embodiments, different numbers and arrangements of platform sections 103 are used, including one, two, four, or more platform sections 103. In some embodiments, one or more of the platform sections 103 can be movable relative to other platform sections 103 and/or relative to the support column 102. For example, in some embodiments, some or all of the platform sections 103 are coupled to adjacent platform sections 103 and/or to the support column 102 by rotatable joints such that at least some of the platform sections 103 can tilt relative to one another and/or relative to the support column 102. The platform assembly 110 can also be movable as a whole relative to the support column 102, as described in greater detail below.

The platform assembly 110 has a longitudinal dimension 198 (see FIG. 1A), a lateral dimension 199 (see FIG. 2) orthogonal to the longitudinal dimension, and a thickness or height dimension (not labeled) orthogonal to both the longitudinal and lateral dimensions. As used herein, the longitudinal dimension 198 refers to a dimension of greatest extent of the platform assembly 110, e.g., when all of the platform sections 103 of the platform assembly are fully extended and all are oriented with their support surfaces roughly aligned in a same plane with one another (or when as close to this state as possible) so as to collectively form a combined support surface that is substantially planar with potentially small gaps between adjacent platform sections 103.

The longitudinal direction extends along the head-to-foot and vice-versa directions. In general, the longitudinal and lateral dimensions 198 and 199 of the platform assembly 110 and the support surfaces of the platform sections 103 are oriented roughly parallel to the ground or other surface on which the table assembly 101 is supported when the platform assembly 110 is in a neutral configuration. For example, in FIGS. 1A-2 the longitudinal dimension 198 is parallel to the x-direction and the lateral dimension 199 is parallel to the y-direction, with the x- and y-directions being parallel to the ground or other surface the table assembly 101 rests upon. Thus, in FIGS. 1A-2, the thickness dimension is parallel to the z-direction, which is perpendicular to the ground or other surface.

However, one of ordinary skill in the art would understand that the platform assembly 110 as a whole and/or individual platform sections 103 thereof do not necessarily have to be parallel to the ground, and that one or both of the longitudinal and/or lateral dimensions 198 and 199 can be tilted relative to the ground in various configurations through which the platform assembly 110 and/or platform section 103 can be movable, including in a neutral configuration in some cases. As used herein, a longitudinally extending side of the platform assembly refers to either of the sides that are parallel to the longitudinal dimension 198. An laterally extending side of the platform assembly refers to either of the sides that are parallel to the lateral dimension 199.

At least one of the platform sections 103 is directly coupled to and supported by the support column 102. The remaining platform sections 103 can be coupled directly to the support column 102 or indirectly to the support column 102 via a chain of one or more intervening platform sections 103. For example, in some embodiments a main platform section 103 (e.g., a middle section 103_2) is coupled to and directly supported by the support column 102 and the others of the platform sections 103 (e.g., end sections 103_1 and 103_3) are coupled to the main platform section 103 or to another platform section 103. As another example, in some embodiments multiple platform sections 103 (all in some embodiments) are coupled directly to the support column 102 and not to another platform section 103.

In some embodiments, some (all, in some cases) of the above-described parts of the table assembly 101 are movable relative to one another. For example, in some embodiments the platform assembly 110 as a whole can be moved relative to the support column 102, such as by tilting around a horizontal axis, swiveling around a vertical axis, translating vertically along the support column 102, translating horizontally relative to the support column 102, and so on. In some embodiments, such movement of the platform assembly 110 as a whole is provided by one or more joints that couple a main platform section 103 (e.g., a middle section 103_2) to the support column 102. Furthermore, as already noted above, individual platform sections 103 can be movable relative to one another and relative to the support column 102 as well, which can be facilitated by joints coupling the platform sections 103 to the support column 102 or to adjacent platform sections 103.

In some embodiments, the platform assembly 110 also includes one or more accessory rails 104. The accessory rails 104 can be configured to receive accessory devices removably mounted thereon, such as arm supports, leg supports, body restraints, bed extensions, various clamps for surgical retractors, and other device holders. In some embodiments, the accessory rails 104 adhere to industry standard specifications familiar to those of ordinary skill in the art to allow compatibility with accessory devices compliant with the standard. The accessory rails 104 can be attached along longitudinally extending sides of one or more of the platform sections 103. One or more openings can be defined between an accessory rail 104 and the side of the platform section 103 to which the accessory rail 104 is attached and portions of accessories mounted to the accessory rail 104 can be inserted through the openings.

As noted above, the system 100 includes one or more manipulators 140. FIGS. 1A-2 illustrate two manipulators 140, but any number of manipulators 140 can be included (such as, for example, one, two, three, or more manipulators mounted to each rail assembly 120, as described in further detail below). A manipulator includes a kinematic structure of links coupled together by one or more joints. The manipulator 140 is movable through various degrees of freedom of motion provided by the joints, thus allowing an instrument 150 mounted thereon to be moved relative to the worksite. For example, some joints can provide for rotation of links relative to one another, other joints can provide for translation of links relative to one another, and some can provide for both rotation and translation. Some or all of the joints can be powered joints, meaning a powered drive element can control movement of the joint through the supply of motive power. Such powered drive elements include, but are not limited to, for example, electric motors, pneumatic or hydraulic actuators, etc. Additionally, some joints can be unpowered joints. The specific number and arrangement of links and joints is not limited. The more links and joints are included, the greater the degrees of freedom of movement of the manipulator 140. A proximal end portion of each manipulator 140 is movably coupled to the table assembly 101 via a rail assembly 120, as described in further detail below.

The instrument 150 can be removably mounted or permanently affixed to the manipulator 140 via an interface. The instruments 150 can include any tool or instrument, including for example industrial instruments and medical instruments (e.g., surgical instruments, imaging instruments, diagnostic instruments, therapeutic instruments, etc.). In embodiments in which the instrument 150 is removably mountable to the manipulator 140, the manipulator 140 can include an instrument manipulator mount (not illustrated) to which the instrument can be removably coupled. The instrument manipulator mount can be located, for example, at a generally distal end portion of the manipulator 140. The manipulator mount has an interface (not illustrated) including output couplers (not illustrated) to engage (directly or indirectly via an intermediary) with input couplers (not illustrated) of the instrument 150 to provide driving forces or other inputs to the mounted instrument 150 to control various degree of freedom movement and/or other functionality of the instrument 150, such as moving an end-effector of the instrument, opening/closing jaws, driving translation and/or rotation of a variety of components of the instrument, delivery of substances and/or energy from the instrument, and various other functions those of ordinary skill in the art are familiar with.

The output couplers can be driven by actuators (e.g., electrical servo-motors, hydraulic actuators, pneumatic actuators) with which those of ordinary skill in the art have familiarity. An instrument sterile adaptor (ISA) can be disposed between the instrument 150 and the instrument manipulator mount interface to maintain sterile separation between the instrument 150 and the manipulator 140. The instrument manipulator mount can also include other interfaces (not illustrated), such as electrical interfaces to provide and/or receive electrical signals to/from the instrument 150. In some embodiments, the system 100 can include flux delivery transmission capability as well, such as, for example, to supply electricity, fluid, vacuum pressure, light, electromagnetic radiation, etc. to the end effector. In other embodiments, such flux delivery transmission can be provided to an instrument through another auxiliary system 1008, described further below and as those of ordinary skill in the art would be familiar with in the context of computer-assisted, teleoperated medical systems.

In some embodiments, the manipulators 140 can be similar to the manipulators described in U.S. Patent Application No. 63/336,778 entitled “NESTING PROXIMAL LINKS FOR TABLE MOUNTED MANIPULATOR SYSTEM, AND RELATED DEVICES, SYSTEMS AND METHODS,” first named inventor Brain Lambrecht, and in U.S. Patent Application No. 63/336,840 entitled “TABLE-MOUNTED MANIPULATOR SYSTEM, AND RELATED DEVICES, SYSTEMS AND METHODS,” first named inventor Steven Manuel, both filed on a date even herewith, or those described in, for example, U.S. Pat. No. 9,358,074 (filed May 31, 2013) to Schena et al., entitled “Multi-Port Surgical Robotic System Architecture,” U.S. Pat. No. 9,295,524 (filed May 31, 2013) to Schena et al., entitled “Redundant Axis and Degree of Freedom for Hardware-Constrained Remote Center Robotic Manipulator,” and U.S. Pat. No. 8,852,208 (filed Aug. 12, 2010) to Gomez et al., entitled “Surgical System Instrument Mounting,” the contents of each of which are incorporated herein by reference in their entirety. Various other embodiments of manipulators can include those as configured as part of the medical systems that are part of various da Vinci® Surgical Systems, such as the da Vinci X®, da Vinci Xi®, and da Vinci SP systems, commercialized by Intuitive Surgical, Inc., of Sunnyvale, California.

As shown in FIGS. 1A-2, the manipulators 140 are coupled to the table assembly 101 via the at least one rail assembly 120. In some embodiments, multiple similar rail assemblies 120 are provided, for example one for each longitudinally extending side of the platform assembly 110. For example, in some embodiments, a first rail assembly 120 can be provided at a first longitudinally extending side of the platform assembly 110 and a second rail assembly 120 can be provided at a second longitudinally extending side of the platform assembly 110. In such embodiments with multiple rail assemblies 120, manipulators 140 can be coupled to the rail assemblies 120 in any number or combination. With the rail assemblies 120 positioned along multiple sides of the platform assembly 120, the manipulators 140 too are positioned along multiple sides of the platform assembly 140. The description below will describe one rail assembly 120 to simplify the description, but the other rail assemblies 120 (if present) can be configured similarly.

The rail assembly 120 includes a movable rail 121 (“rail 121”), one or more movable manipulator carriages 126 (two being shown in the embodiment of FIGS. 1A-2) coupled to the rail 121 and to the manipulators 140 (e.g., at least one carriage 126 can be provided per manipulator 140), and one or more rail mounting carriages 127 coupled to the rail 121 and the table assembly 101. In some embodiments, the rail assembly 120 further includes a fixed rail 124, which can be coupled between the rail mounting carriages 127 and the table assembly 101 (either to the platform assembly 110 or alternatively, as depicted by the dashed structure 124′ in FIG. 2, to the support column 102), while in other embodiments the rail mounting carriages 127 can be coupled directly to the table assembly 101.

Each movable manipulator carriage 126 is moveable along a longitudinal dimension 197 of the rail 121 and couples a respectively corresponding one of the manipulators 140 to the rail 121 such that the manipulators 140 can translate relative to the rail 121 along the longitudinal dimension 197 of the rail 121. In FIGS. 1A-2, one movable manipulator carriage 126 is shown per manipulator 140, but multiple movable manipulator carriages 126 could be provided to operably couple to and support a given manipulator 140. In some embodiments, the longitudinal dimension 197 of the rail 121 is parallel to the longitudinal dimension 198 of the platform assembly 110 (e.g., parallel to the x-axis) in a neutral configuration of the platform assembly 110, as shown in FIG. 1A. The movable rail 121 includes a first set of engagement features 122 configured to engage with complementary engagement features 128 of the movable manipulator carriages 126. For example, as shown in FIG. 2, the first set of engagement features 122 of the rail 121 includes a track including flanges extending along the longitudinal dimension 197, and the complementary engagement features 128 of the carriages 126 are configured to engage and ride along the flanges of the first set of engagement features 122. The first set of engagement features 122 can also include a track including grooves in which the complementary engagement feature 128 are received.

Any other type of complementary engagement features that when engaged allow for relative translation can be used as the complementary engagement features 122 and 128, and those having ordinary skill in the art are familiar with various complementary engagement features that are used in rail and carriage systems. In some embodiments, the first set of engagement features 122 and/or the complementary engagement features 128 include bearing devices configured to reduce friction to facilitate easier translation, such as wheels, balls, plain bearing surfaces coated or otherwise provided with a low friction material, and other friction reducing mechanisms.

The one or more rail mounting carriages 127 couple the rail 121 to the table assembly 101 such that the rail 121 can translate relative to the platform assembly 110 and/or support column 102 along directions of the longitudinal dimension 197 of the rail 121. This translation between the rail 121 and the platform assembly 110 and/or support column 102 is provided at least in part by a movable coupling between the rail mounting carriages 127 and the rail 121, such that the rail 121 and the second carriages 127 can translate relative to one another along the directions of the longitudinal dimension 197 of the rail 121. In addition, in some embodiments, the rail mounting carriages 127 are also movably coupled to the table assembly 101 such that the second carriages 127 are translatable relative to the table assembly 101 along the directions of the longitudinal dimension 197 of the rail 121. Thus, in these embodiments, translation of the rail 121 relative to the table assembly 101 can occur through either one or both of relative translation between the carriages 127 and the table assembly 101 and relative translation between the carriages 127 and rail 121.

For example, in some embodiments a first end of the rail mounting carriage 127 is movably coupled to a fixed rail 124, which in turn is attached to one of the platform sections 103 or to the support column 102 (the latter shown in dashed at 124′), and the rail mounting carriage 127 is translatable along the fixed rail 124. However, in other embodiments the first end of the rail mounting carriage 127 is fixedly attached to or is part of the platform assembly 110 and/or support column 102, and thus the carriages 127 remain stationary relative to the platform assembly 110 and/or support column 102 (in such embodiments, the fixed rail 124 is omitted).

Regardless of whether the first end of the rail mounting carriage 127 is fixedly or movably coupled to the platform assembly 110 and/or support column 102, in some embodiments a second end of the rail mounting carriage 127 is moveably coupled to the movable rail 121 such that the rail 121 and the carriage 127 are translatable relative to one another. In particular, the rail includes a second set of engagement features 123 (e.g., tracks or other engagement features) that engage with complementary engagement features 125 of the carriage 127 to couple the rail 121 to the carriage 127 while allowing translation between the rail 121 and carriage 127. The movability of the carriages 127 relative to the table assembly 101 can allow for a greater range of motion of the manipulators 140 and/or for a shorter length of the rail 121, as compared to a configuration in which the rail 121 is fixed relative to the table assembly 101. This can also enable the rail assembly 120 to more easily be moved out of the way of the platform assembly 110 to avoid interference therewith as the platform assembly 110 and/or individual platform sections 103 thereof are moved through various configurations.

In other embodiments, the rail mounting carriages 127 are stationary relative to the table assembly 101 and translation of the rail 121 relative to the table assembly 101 occurs solely through relative translation between the carriages 127 and the rail 121. For example, in some embodiments a first end of a rail mounting carriage 127 is attached to one of the platform sections 103 or to the support column 102 and a second end of the second carriage is moveably attached to the movable rail 121. One rail mounting carriage 127 is shown in FIGS. 1A-2 for ease of description, but any number could be used.

As noted above, in some embodiments the one or more rail mounting carriages 127 are coupled (directly or via an intermediary such as the fixed rail 124, 124′) to one of the platform sections 103 or to the support column 102. Which structure the carriages 127 are coupled to can make a difference in embodiments in which the platform assembly 110 as a whole is movable relative to the support column 102, for example by tilting relative to the support column 102. In embodiments in which the rail mounting carriages 127 are coupled to one of the platform sections 103 (e.g., a middle section 103_2) (either directly or via an intermediary such as fixed rail 124), when the platform assembly 110 moves relative to the support column 102, the rail assembly 120 and hence the manipulators 140 coupled thereto move along with the platform assembly 110. This can allow the manipulators 140 to automatically maintain a set pose and position relative to the platform assembly 110, and thus relative to a patient supported on the platform assembly, regardless of a configuration of the platform assembly 110 and without having to reposition the manipulators 140. Moreover, in some circumstances, collision between the platform assembly 110 and the rail assembly 120 due to motion of the platform assembly 110 can be avoided as they both move together.

In other embodiments in which the rail mounting carriages 127 are coupled to the support column 102 (either directly or via an intermediary such as the fixed rail 124′, when the platform assembly 110 moves relative to the support column 102, the rail assembly 120 and hence the manipulators 140 coupled thereto remain with the support column 102 and do not move along with the platform assembly 110. This can allow greater motion of the rail assembly 120 and manipulators 150 relative to the platform assembly 110. This also can increase the strength of the structure between the manipulators 150 and the support column 102 by reducing the length of the structure between them and reducing the number of connections between them.

In some embodiments, motors or other actuation devices (not illustrated) are provided to drive the relative translation between the rail 121 and the carriages 126, between the rail 121 and the carriages 127, and/or between the carriages 127 and the table assembly 101. In some embodiments, motors/actuators are housed within the rail 121. In some embodiments, motors/actuators are housed within the carriages 126 and/or 127.

FIGS. 1A-1C show the rail assembly 120 in various configurations illustrative of a range of motion provided by the rail assembly 120 in some embodiments. In FIG. 1A, the rail mounting carriage 127 is positioned at a middle portion of the movable rail 121 and thus the movable rail 121 is positioned near a middle portion of the table assembly 101 under the middle section(s) 103_2. In this configuration, the manipulators 140 can be positioned in a variety of positions, for example, along the middle section(s) 103_2 of the platform assembly 110, which can be useful for performing a variety of operations. Moreover, in this configuration the rail assembly 120 and manipulators 140 are positioned away from the two end regions (head and foot end regions) of the platform assembly 110, which can beneficially free up space at these regions and thus allow personnel or other equipment to be positioned at, near, or under the end regions of the platform assembly 110.

In addition, in some embodiments, one or both of the end platform sections 103_1 and 103_3 could be lowered (declined) relative to the middle section 103_2 while the rail assembly 120 is in this configuration because in some instances the rail 121 does not protrude into the space that would be occupied by the lowered end sections 103_1 or 103_3 and hinder those section from lowering. Whether the rail 121 protrudes into the space occupied by the end sections 103_1 or 103_3 depends on the sizes (lengths) of the rail 121 and platform assembly 110 as well as on the positioning of the rail 121.

In FIG. 1B, a foot end portion of the movable rail 121 and thus the movable rail 121 is positioned near a foot end of the table assembly 101 under the second end section 103_3. That is, to reach the state shown in FIG. 1B from the state illustrated in FIG. 1A, the rail 121 is translated longitudinally toward the foot end of the table assembly relative to the rail mounting carriage 127 until a head end portion of the rail 121 reaches the carriage 127. In embodiments in which the rail mounting carriage 127 is also movable relative to the fixed rail 124, the carriage 127 can also be translated toward the foot end of the fixed rail 124 (both translation of the rail 121 relative to the second carriage and of the second carriage 127 relative to the rail 124 can occur simultaneously, or in any order, as desired). In this configuration, the manipulators 140 can be positioned, for example, along the longitudinally extending side 109b of the platform assembly 110 near the second end section 103_3 of the platform assembly 110 and near some portions of the middle section 103_2. Moreover, because the rail assembly 120 is moved away of the first end section 103_1 in this configuration, the first end section 103_1 can be placed in a declined position relative to the middle section 103_1 without interference and/or personnel or other equipment could be positioned at/near/under the first end section 103_1.

Furthermore, in some embodiments, the positioning of the rail 121 shown in FIG. 1B allows the manipulators 140 to be moved in a longitudinal (x-axis) direction beyond the foot end of the platform assembly 110, which can enable the manipulators 140 to further be moved in a laterally inward direction (positive y-axis direction) such that the manipulators 140 are positioned along the head end, laterally extending side 109a of the platform assembly 110 instead of along the longitudinally extending side 109b of the platform assembly 110. Thus, in this position the manipulators 140 are out of the way of and do not obstruct the longitudinally extending side 109b of the platform assembly 110. Such positioning can be desirable in various stages of a medical procedure, such as, for example, transferring a patient from a gurney to the platform assembly 110. Moreover, in this position the manipulators 140 remain in the sterile field and thus can be moved out of the way for a task (e.g., patient transfer) while remaining covered with a sterile drape prior to the task (e.g., patient transfer).

In FIG. 1C, the rail mounting carriage 127 is positioned at a foot end portion of the movable rail 121 (right end of movable rail 121 in FIG. 1C) and therefore the rail 121 is positioned near the head end of the table assembly 101 under the first end section 103_1. That is, to reach the state shown in FIG. 1C from the state illustrated in FIG. 1A, the rail 121 is translated longitudinally toward the head end of the table assembly 101 relative to the carriage 127. In embodiments in which the carriage 127 is also movable relative to the fixed rail 124, the carriage 127 can also be translated toward the head end of the fixed rail 124 (both translation of the rail 121 relative to the second carriage and of the carriage 127 relative to the rail 124 can occur simultaneously, or in any order, as desired). In this configuration, the manipulators 140 can be positioned, for example, along the head end, longitudinally extending side 109b of the table assembly 101 near the first end section 103_1 of the platform assembly 110 and near portions of the middle section 103_2. Moreover, because the rail assembly 120 is moved away of the second end section 103_3 in this configuration, the second end section 103_3 can be placed in a declined position relative to the middle section 103_2 without interference and/or personnel or other equipment could be positioned at/near/under the second end section 103_3.

In some embodiments (not illustrated), the positioning of the rail 121 allows the manipulators 140 to be moved in a longitudinal (x-axis) direction beyond the head end of the platform assembly 110 and then in a lateral (y-axis) direction such that the manipulators 140 are positioned along the laterally extending side 109a of the head end of the platform assembly 110 instead of along the longitudinally extending side 109b of the platform assembly 110, similar to the positioning described above in relation to FIG. 1B except at the head end of the platform assembly rather than the foot end. In some embodiments, the manipulators 140 can be moved beyond both the head and foot ends of the platform assembly 110 in the above-described manner, in other embodiments the manipulators 140 can be moved beyond the head end only, and in still other embodiments can be moved beyond the foot end only.

Thus, the rail assembly 120 of the embodiment of FIGS. 1A-2 provides a wide range of motion for the manipulators 140, which can allow the manipulators 140 to be positioned nearly anywhere along the length of the platform assembly 110. Moreover, not only does the rail assembly 120 provide a wide range of motion for the manipulators 140, it does so while also minimizing interference between the rail assembly 120 and the platform assembly 110, personnel, and other equipment. In particular, the rail assembly is movable between different positions based on the configuration of the platform assembly 110 and/or based on various tasks during a medical procedure that can benefit from the rail 121 positioned out of the way of the portions of the platform assembly 110 (e.g., out of the way of a platform section 103 being lowered), personnel, or other equipment as needed.

Furthermore, the rail assembly 120 can be configured to remain within an overall envelope 108 defined by the platform assembly 110, which corresponds to a footprint or peripheral profile of the platform assembly 110 when viewed from above (i.e., a projection of the platform assembly 110 onto the plane of the ground surface), throughout all motion of the rail 121 relative to the platform assembly 110 and/or support column 102 (i.e., an entirety of the rail is within the envelope 108). This further reduces the degree of interference between the rail assembly 120 and other equipment, as will be described in greater detail below. The envelope 108 includes longitudinal boundaries 108a and lateral boundaries 108b defined by outer laterally extending sides 109a and outer longitudinally extending sides 109b of the platform assembly 110. The outer longitudinal and lateral boundaries 108a, 108b of the envelope 108 correspond to vertical projections from the aforementioned outer laterally and longitudinally extending sides 109a and 109b of the platform assembly 110, respectively, down to the ground or other surface upon which the table assembly 101 rests. For example, in embodiments in which the platform assembly 110 has a rectangular profile, as shown in FIG. 1A, the longitudinal boundaries 108a of the envelope 108 correspond to vertical planes that are perpendicular to the longitudinal dimension 198 (i.e., perpendicular to the x-axis) and tangent to the laterally extending sides 109a, and similarly, as shown in FIG. 2, the lateral boundaries 108b of the envelope 108 correspond to vertical planes that are perpendicular to the lateral dimension 199 (i.e., perpendicular to the y-axis) and tangent to the longitudinally extending sides 109b. The envelope 108 can also include upper and lower boundaries (not labeled) corresponding, respectively, to a top surface of the platform assembly 110 and to the ground (or other surface upon which the table assembly 101 rests).

As shown in FIG. 2, in some embodiments the laterally extending sides 109a of the platform assembly 110 that defines the longitudinal boundaries 108a of the envelope 108 is the outermost edge of the platform assembly 110 excluding the accessory rail 104, if present. Thus, in such embodiments, the envelope 108 defined by the platform assembly 110 is contained laterally inside of the accessory rails 104, if present. This can be beneficial in some circumstances because it ensures that the rail assembly 120 will not interfere with any accessories that are mounted to the accessory rail 104. In particular, because the rail assembly 120 remains within the envelope 108 throughout the entire range of motion of the rail 121, and because the envelope 109 is laterally inside of the accessory rails 104 in these embodiments, the rail 121 or other portions of the rail assembly 120 cannot collide with or otherwise interfere with the accessories. Moreover, even in embodiments in which the rail 104 is not present or when the envelope 108 extends to the edge of the rail 104, the rail assembly 120 remaining within the envelope 108 of the platform assembly 110 still helps to reduce interference by the rail assembly 120 with other equipment or personnel positioned around the table assembly 101, as it can be known in advance that the rail assembly 120 will not collide with or otherwise interfere with equipment or personnel that are positioned outside of the envelope 108.

Although embodiments are described above in which the envelope 108 includes both longitudinal and lateral boundaries 108a, 108b and the rail assembly 120 remains within all of these boundaries throughout its range of motion, in other embodiments the rail assembly 120 can remain within only a subset of these boundaries. For example, in some embodiments, the rail assembly 120 remains within the lateral boundaries 108b, but not necessarily within the longitudinal boundaries 108a (e.g., the rail assembly 120 can be moved to be positioned to at least partially extend beyond the longitudinal boundaries 108a (e.g., beyond a head or foot end of the platform assembly) while remaining within the lateral boundaries 108b in some embodiments).

Returning to FIG. 1A, the user input and feedback system 1004, control system 1006, and auxiliary system 1008 will be described. Some or all of these components can be provided at a location remote from the table assembly 101. The user input and feedback system 1004 is operably coupled to the control system 1006 and includes one or more input devices to receive input control commands to control operations of the manipulators 140, instruments 150, rails assembly 120, and/or table assembly 101. Such input devices can include but are not limited to, for example, telepresence input devices, triggers, grip input devices, buttons, switches, pedals, joysticks, trackballs, data gloves, trigger-guns, gaze detection devices, voice recognition devices, body motion or presence sensors, touchscreen technology, or any other type of device for registering user input. In some cases, an input device can be provided with the same degrees of freedom as the associated instrument that they control, and as the input device is actuated, the instrument, through drive inputs from the manipulator assembly, is controlled to follow or mimic the movement of the input device, which can provide the user a sense of directly controlling the instrument. Telepresence input devices can provide the operator with telepresence, meaning the perception that the input devices are integral with the instrument. The user input and feedback system 1004 can also include feedback devices, such as a display device (not shown) to display images (e.g., images of the workspace as captured by one of the instruments 1010), haptic feedback devices, audio feedback devices, other graphical user interface forms of feedback, etc.

The control system 1006 can control operations of the system 100. In particular, the control system 1006 can send control signals (e.g., electrical signals) to the table assembly 101, rail assembly 120, manipulators 140, and/or instruments 150 to control movements and/or other operations of the various parts. In some embodiments, the control system 1006 can also control some or all operations of the user input and feedback system 1004, the auxiliary system 1008, or other parts of the system 100. The control system 1006 can include an electronic controller to control and/or assist a user in controlling operations of the manipulator assembly 1001. The electronic controller includes processing circuitry configured with logic for performing the various operations. The logic of the processing circuitry can include dedicated hardware to perform various operations, software (machine readable and/or processor executable instructions) to perform various operations, or any combination thereof.

In configurations in which the logic includes software, the processing circuitry can include a processor to execute the software instructions and a memory device that stores the software. The processor can include one or more processing devices capable of executing machine readable instructions, such as, for example, a processor, a processor core, a central processing unit (CPU), a controller, a microcontroller, a system-on-chip (SoC), a digital signal processor (DSP), a graphics processing unit (GPU), etc. In cases in which the processing circuitry includes dedicated hardware, in addition to or in lieu of the processor, the dedicated hardware can include any electronic device that is configured to perform specific operations, such as an Application Specific Integrated Circuit (ASIC), a Field Programmable Gate Array (FPGA), a Complex Programmable Logic Device (CPLD), discrete logic circuits, a hardware accelerator, a hardware encoder, etc. The processing circuitry can also include any combination of dedicated hardware and processor plus software.

Differing degrees of user control versus autonomous control can be utilized in the system 100, and embodiments disclosed herein can encompass fully user-controlled systems, fully autonomously-controlled systems, and systems having any combination of user and autonomous control. For operations that are user-controlled, the control system 1006 generates control signals in response to receiving a corresponding user input command via the user input and feedback system 1004. For operations that are autonomously controlled, the control system 1006 can execute pre-programmed logic (e.g., a software program) and can determine and send control commands based on the programming (e.g., in response to a detected state or stimulus specified in the programming). In some systems, some operations can be user controlled and others autonomously controlled. Moreover, some operations can be partially user controlled and partially autonomously controlled—for example, a user input command can initiate performance of a sequence of events, and then the control system 1006 can perform various operations associated with that sequence without needing further user input.

In some embodiments, the control system 1006 can control positions of the manipulators 140 and the positions or configurations of the rail assembly 120 and/or the table assembly 101 by sending electrical signals to drive actuators that move the manipulators 140, the rail assembly 120, and/or the table assembly 101. In some embodiments, the control system 1006 can determine states or configurations of the table assembly 101 and change a configuration of the rail assembly 120 based on the determined state or configuration of the table assembly 101. For example, in response to determining that the table assembly 101 is going to be transitioned to a state/configuration in which interference between the rail assembly 120 and parts of the table assembly 101 might occur, the control system 1006 can automatically move the rail assembly 120 into a predetermined configuration in which the rail assembly 120 will not interfere with the table assembly 101. For example, if the second end portion 103_3 is going to be moved into a declined position, the control system 1006 can move the rail 121 toward the first end portion 103_1 (e.g., the configuration shown in FIG. 1C) so that the rail 121 is out of the way of the second end portion 103_3. The control system 1006 can determine that a transition to a given state/configuration of the table assembly 101 is occurring or is about to occur by, for example, detecting one or more sensor signals indicative that the transition is occurring (e.g., from a sensor configured to sense motion/position of a platform section 103 or other portion of the table assembly 101) or by receiving input or signals associated with a request/command for the state transition to occur (e.g., in response to a user pressing a button to cause the state change).

The auxiliary system 1008 can include various auxiliary devices that can be used in operation of the system 100. For example, the auxiliary system 1008 can include power supply units, auxiliary function units (e.g., functions such as irrigation, evacuation, energy supply, illumination, sensors, imaging, etc.). As one example, in a system 100 for use in a medical procedure context, the auxiliary system 1008 can include a display device for use by medical staff assisting a procedure, while the user operating the input devices can utilize a separate display device that is part of the user input and feedback system 1004. As another example, in a system 100 for use in a medical context, the auxiliary system 1008 can include flux supply units that provide surgical flux (e.g., electrical power) to instruments 150. An auxiliary system 1008 as used herein can thus encompass a variety of components and does not need to be provided as an integral unit.

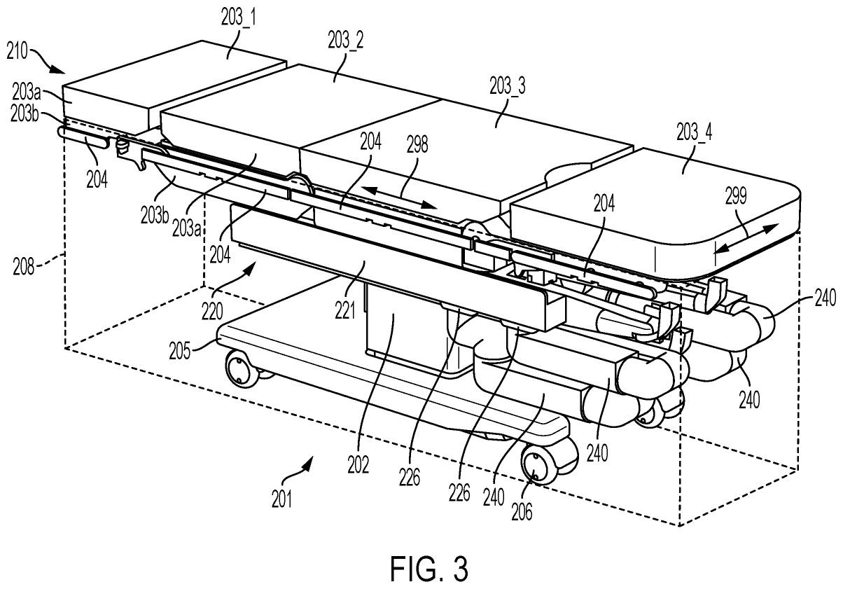

Turning now to FIGS. 3-5D, a table-mounted manipulator system 200 (“system 200”) in accordance with another embodiment is illustrated. The system 200 can be used as the system 100, and some components of the system 200 can be used as components of the system 100 described above. Thus, the descriptions of the components of the system 100 above are applicable to the related components of the system 200, and duplicative descriptions of these components are omitted below. The related components of the systems 100 and 200 are given reference numbers having the same right-most two digits—for example, 105 and 205. Although the system 200 is one embodiment of the system 100, the system 100 is not limited to the system 200.

As shown in FIGS. 3-5D, the system 200 includes a table assembly 201, two rail assemblies 220 coupled to the table assembly, and multiple manipulators 240 coupled to the rail assemblies 220. Each manipulator 240 carries one more instruments (not illustrated), which can be removably or permanently mounted thereon, as described above. The system 200 also can include a control system (not illustrated), a user input and feedback system (not illustrated), and/or an auxiliary system (not illustrated) similar to those described above in relation to the system 100. In some embodiments, the system 200 is configured as a computer-assisted, teleoperable medical system. In other embodiments, the system 200 is configured as a teleoperable system for use in non-medical contexts.

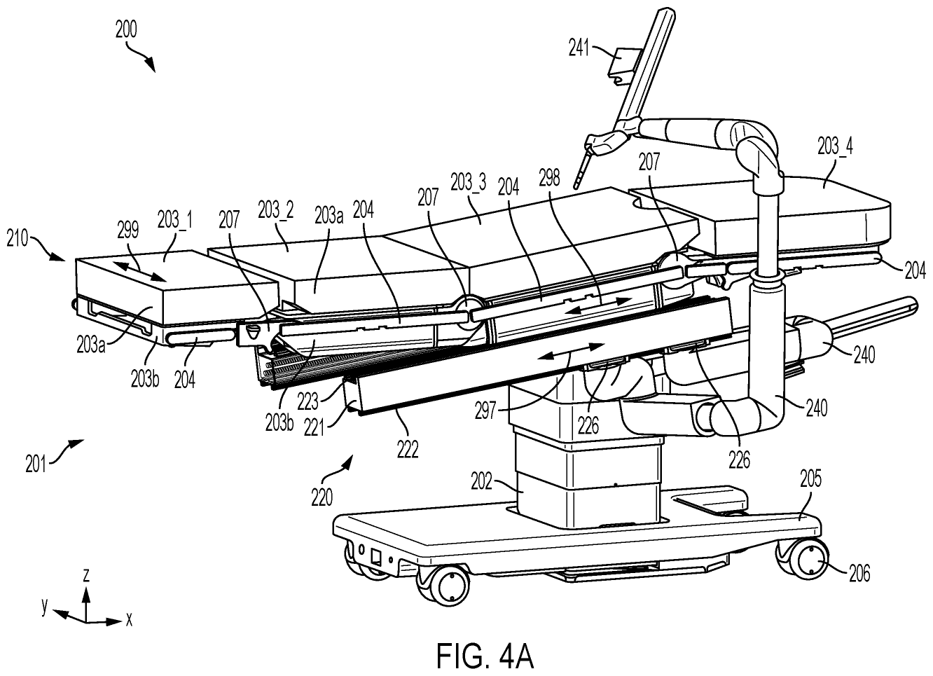

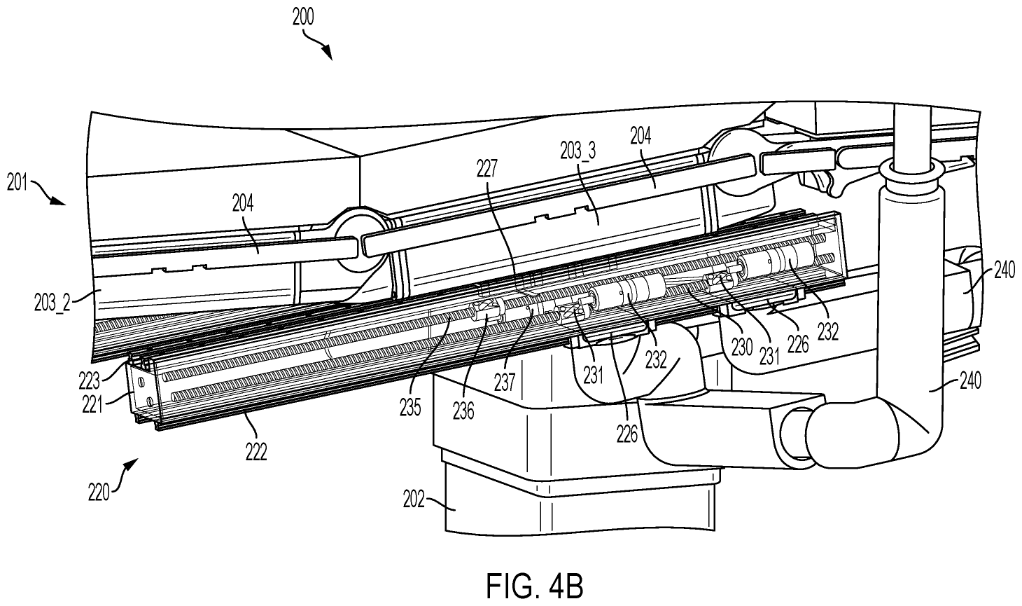

As shown in FIGS. 3 and 4A, the table assembly 201 includes a platform assembly 210 configured to support the patient or inanimate workpiece, a support column 202 coupled to and supporting the platform assembly 210, and a base 205 coupled to the support column 202. The base 205 can contact the ground or other surface upon which the table assembly 201 rests, and in some embodiments the base 205 includes wheels 206 (or other mobility devices as described above) to allow movement of the system 200 along the ground or other surface. As shown in FIG. 4A, in some embodiments, the support column 202 includes a telescoping support column that can raise or lower the platform assembly 210.

The platform assembly 210 includes multiple platform sections 203 configured to support the patient or workpiece. In particular, in the embodiment illustrated in FIGS. 3-5, the platform assembly 210 includes first end section 203_1 (“head section 203_1”), middle sections 203_2 and 203_3, and second end section 203_4 (“foot section 203_4”), which are arranged in series and movably coupled together via joints 207. In some embodiments, the first end section 203_1 is configured to support a head of the patient, the second end section 203_4 is configured to support the feet and/or legs of the patient, and the more middle sections 203_2 and 203_3 are configured to support a torso and/or other portions of the patient. The joints 207 allow adjacent platform sections 203 to pivot relative to one another about rotation axes parallel to a lateral dimension 299 of the platform assembly 210 (e.g., parallel to a y-axis in the Figures).

FIG. 3 illustrates the platform assembly 210 in a neutral configuration in which all of the platform sections 203 are parallel to one another, and FIG. 4A illustrates the platform assembly 210 in an articulated configuration in which some of the platform sections 203 are oriented at non-zero angles relative to adjacent platform sections 203. In some embodiments, some of the joints 207 also allow for other motion between adjacent platform sections 203, such as relative translation along the longitudinal dimension 298 or relative rotation around a vertical axis parallel to a height dimension (i.e., the z-axis in the Figures), which is perpendicular to the lateral and longitudinal dimensions 299 and 298. In some embodiments, the platform sections 203 include relatively rigid support portions 203b and softer cushion portions 203a attached to the support portions 203b, with a surface of the cushion portions 203a (i.e., the top surface in the orientation illustrated in FIG. 4) forming a support surface that contacts the patient or workpiece. In some embodiments, multiple platform sections 203 can share some components. For example, as illustrated in FIG. 3, the middle platform sections 203_2 and 203_3 can share the same cushion portion 203a that extends across both platform sections 203_2 and 203_3. The cushion portion 203a shared by the platform sections 203_2 and 203_3 can bend when the platform sections 203_2 and 203_3 are articulated relative to one another, as shown in FIG. 4A.

In addition to moving individual platform sections 203 relative to adjoining platform sections 203, the platform assembly 210 as a whole is movable relative to the support column 202. In some embodiments, the middle section 203_3 is coupled to the support column 202 by one or more joints (not illustrated), providing for motion between the middle section 203_3 and the column 202. The other platform sections 203_1, 203_2, and 203_4 are coupled (directly or indirectly) to the middle section 203_3, and thus as the middle section 203_3 moves relative to the support column 202 the platform assembly 210 as a whole moves relative to the support column 202.

In some embodiments, the motion of the middle section 203_3 (and hence platform assembly 210 as a whole) relative to the support column 202 includes pivoting (tilting) about a horizontal axis parallel to the lateral dimension 299 (e.g., a pitch degree of freedom of motion), as shown in FIG. 4A. In some embodiments, other degrees of freedom of motion are provided between the middle section 203_3 and the support column 202, including pivoting (tilting) about a horizontal axis parallel to the longitudinal dimension 298 (e.g., a roll degree of freedom of motion), rotating about a vertical axis (e.g., a yaw degree of freedom of motion), and/or translation along the longitudinal and/or lateral dimensions 299 or 298. As shown in FIG. 3, in the neutral configuration of the platform assembly 210, the platform assembly 210 is parallel to the ground (or other surface upon which the system 200 rests)—in particular, the support surfaces of the platform sections 203 are parallel to the ground (or other surface).

As shown in FIGS. 3-5D, the platform assembly 210 also includes a number (one or more) of accessory rails 204 attached to side surfaces of the support portions 203b of the platform sections 203.

As noted above, the system 200 includes multiple manipulators 240. In the embodiment illustrated in FIG. 3, four manipulators 240 are present, with two manipulators 240 on each longitudinal side of the platform assembly 210 (i.e., two manipulators 240 are mounted to a rail assembly 220 along a first longitudinal side of the platform assembly 210 and two manipulators 240 are mounted to a rail assembly 220 along a second longitudinal side of the platform assembly 210, wherein the second longitudinal side is opposite to the first longitudinal side). In other embodiments, more or fewer manipulators 240 can be mounted to rails 221, such as one, two, three, or more manipulators per longitudinal side.

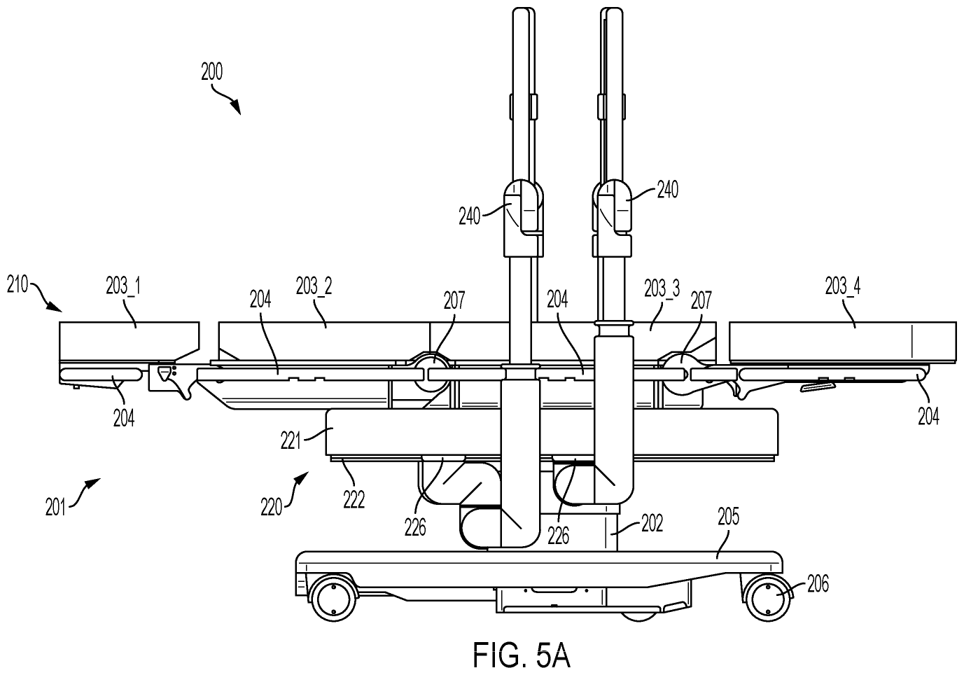

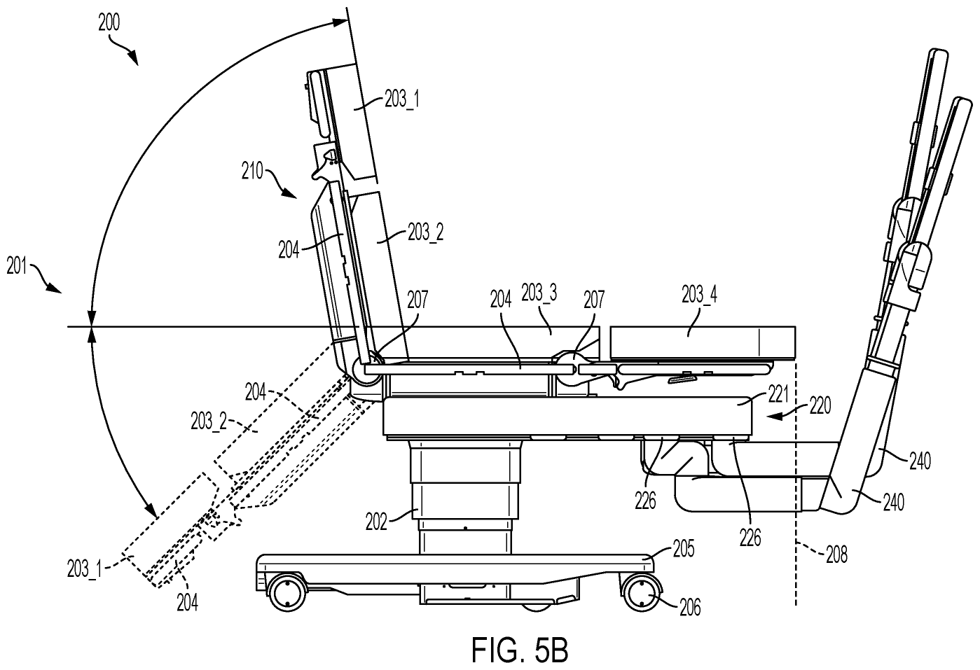

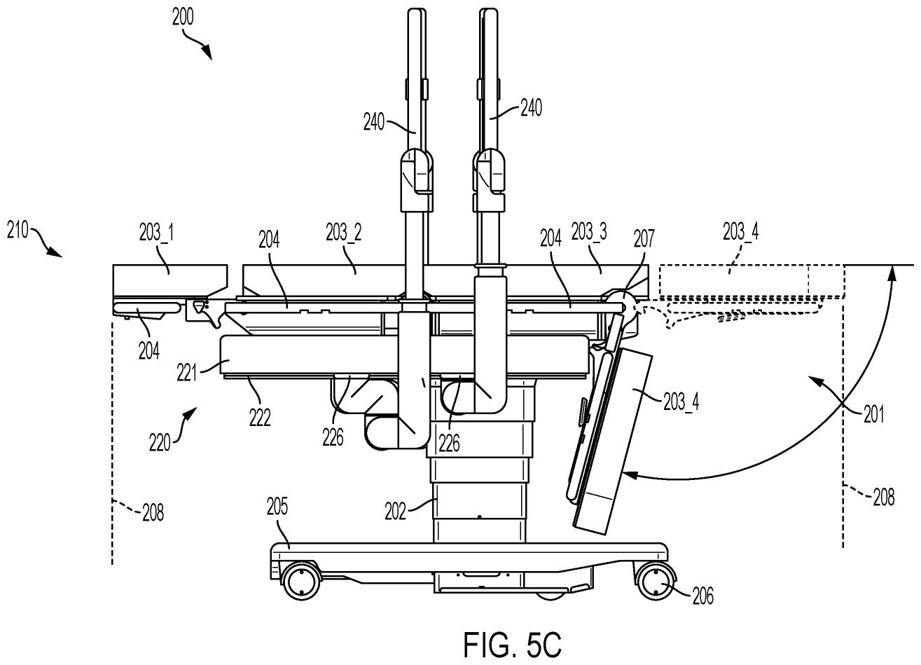

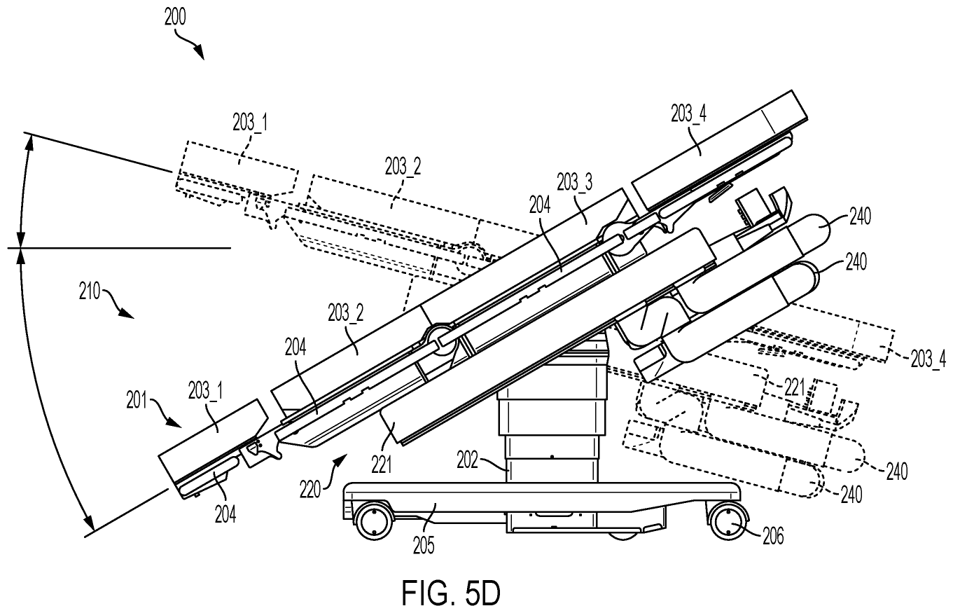

In FIG. 3, the manipulators 240 are shown in a stowed state under end section 203_4, while in FIGS. 4A-5D the manipulators 240 are shown in various deployed states (only the two manipulators 240 disposed on one side of the platform assembly 210 are visible in FIGS. 4A-5D). The deployed states include states in which the manipulators 240 are not stowed, e.g., distal ends of the manipulators 240 are positioned at or above the level of the platform assembly 110, and can include a variety of configurations and positions of the manipulators 240 including but not limited to those shown in FIGS. 4A-5C. The manipulators 240 include a number of links and joints, as well as an instrument manipulator mount 241 configured to receive an instrument removably mounted thereon and including an interface with output couplers to transfer driving force or other inputs to the instrument.

As shown in FIGS. 3-5D, the manipulators 240 are coupled to the table assembly 201 via the rail assemblies 220. One rail assembly 220 is provided for each of the two longitudinally extending sides of the platform assembly 210. The description below describes a single rail assembly 220 for ease of description, and the other rail assembly 220 can be configured similarly. As shown in FIGS. 4A and 4B, the rail assembly 220 includes a movable rail 221 (“rail 221”), movable manipulator carriages 226 coupled to the rail 221 and to the manipulators 240 (at least one movable manipulator carriage 226 per manipulator 240), and one or more rail mounting carriages 227 coupled to the rail 221 and the table assembly 201 (see FIG. 4B).

Each movable manipulator carriage 226 couples a respectively corresponding one of the manipulators 240 to the rail 221 such that the manipulators 240 can translate relative to the rail 221 along a longitudinal dimension 297 of the rail 221. In addition, each rail mounting carriage 227 couples the rail assembly 220 to the platform assembly 210 (e.g., to the middle section 203_3) such that the rail 221 can translate relative to the platform assembly 210 and the support column 202 along the longitudinal dimension 297 of the rail 221. In the embodiment of FIGS. 3-5D, because the rail assembly 220 is coupled to the platform middle section 203_3, the longitudinal dimension 297 of the rail 221 is parallel to the longitudinal dimension 298 of the platform assembly 210 regardless of how the platform assembly 210 is moved or oriented relative to the support column 202.

As shown in FIG. 4B, which depicts interior components of the rail 221, the movable rail 221 includes first and second sets of engagement features 222 and 223 configured to engage with complementary engagement features of the carriages 226 and 227, respectively, to movably couple the rail 221 to the carriages 226 and 227. The first set of engagement features 222 is configured to engage with complementary engagement features of the movable manipulator carriages 226, while the second set of engagement features 223 is configured to engage with complementary engagement features (not visible) of the rail mounting carriages 227, similar to that described with reference to FIGS. 1A-2.

The first and second sets of engagement features 222 and 223 each includes two tracks including flanges and/or grooves extending along the longitudinal dimension 297, and the complementary engagement features of the carriages 226 and 227 are configured to engage and ride along the flanges and/or in the grooves of the first and second sets of engagement features 222 and 223, respectively. The tracks of the first set of engagement features are spaced apart from one another in the lateral dimension to provide increased stability to and strengthen the coupling with the movable manipulator carriages 226. The second set of engagement features 223 is configured similarly. In some embodiments, the complementary engagement features of the carriages 226 and 227 include bearing devices (not illustrated) configured to reduce friction to facilitate easier translation, such as wheels, balls, plain bearing surfaces, etc.

As shown in FIG. 4B, the rail assembly 220 further includes a first translation drive mechanism to provide relative translation between the rail 221 and the movable manipulator carriages 226 and thus the manipulators 240 supported by the rail 221. The first translation drive mechanism includes a lead screw 230 extending along the longitudinal dimension 297 and fixed relative to the rail 221, follower nuts 231 engaged with the lead screw 230, and coupled to the carriages 226, and actuators 232 coupled to the follower nuts 231. The lead screw 230 includes an external threaded surface and the follower nuts 231 include a complementary internal threaded surface engaged with the external threaded surface of the lead screw 230 such that rotation of the follower nuts 231 relative to the lead screw 230 causes translation of the follower nuts 231 along the lead screw 230.

The actuators 232 can be electric motors or other actuation devices (e.g., hydraulic, pneumatic, or other devices to provide a motive force) configured to drive rotation of a corresponding follower nut 231. The actuators 232 are operably coupled to the corresponding follower nut 231, for example, via a gear mechanism or other motion transfer mechanism that converts motion of the actuator 232 into rotation of the follower nut 231. A follower nut 231 is coupled to a corresponding movable manipulator carriage 226 such that the follower nut 231 and carriage 226 are constrained to translate together (i.e., to remain translationally stationary relative to one another) but with the follower nut 231 being rotatable relative to the carriage 226 (i.e., rotatable around the lead screw 230). A coupling mechanism (not visible in FIG. 4B) couples the follower nut 231 to the carriage 226 and extends down from the follower nut 231 through an opening provided in a bottom wall of the rail 221 in the region between the two tracks of the first set of engagement features 222. In some embodiments, instead of or in addition to the lead screw 230, the first translation mechanism can include one or more ball screws, a rack-and pinion drive, a cable/pulley drive system (described in more detail below), or various other devices to actuate and impart translational movement between the carriage 226 and rail 221.

In the embodiment of FIGS. 3-5, the one or more rail mounting carriages 227 couple the rail 221 to the table assembly 201, for example to the middle section 203_3 in the embodiment illustrated in FIGS. 3-5D, such that the rail 221 can also translate relative to the platform assembly 210 and support column 202 in a direction of the longitudinal dimension 297 of the rail 221. This translation between the rail 221 and the platform assembly 210 and/or support column 202 is provided at least in part by the rail 221 translating relative to the rail mounting carriages 227. Specifically, the rail 221 can include a second translation drive mechanism including a second lead screw 235, follower nut 236, and actuator 237, which components are configured and operate similarly to the lead screw 230, follower nut 231, and actuator 232 described above except for being coupled to the rail mounting carriages 227 instead of the movable manipulator carriages 226. The actuator 237 drives rotation of the follower nut 236, which causes translation of the follower nut 236 along the lead screw 235, which in turn causes relative translation between the carriages 227 and the rail 221. Because the carriages 227 are attached to the platform assembly 110 (or alternatively support column), the relative translation between the carriages 227 and the rail 221 causes translation of the rail 221 relative to the platform assembly 110 (i.e., in a reference frame fixed to the platform assembly 110 or to the ground, the carriages 227 are seen as stationary and the rail 221 is seen as translating). In some embodiments, instead of or in addition to the lead screw 235, the second drive translation mechanism can include one or more ball screws, a rack-and-pinion drive, a cable/pulley drive system (described in more detail below), or various other devices to actuate and impart translational movement between the carriage 227 and rail 221.

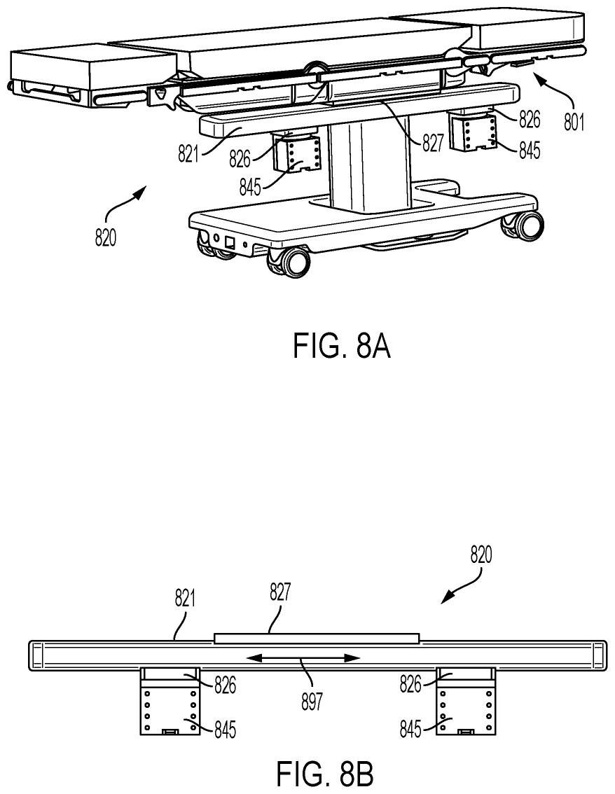

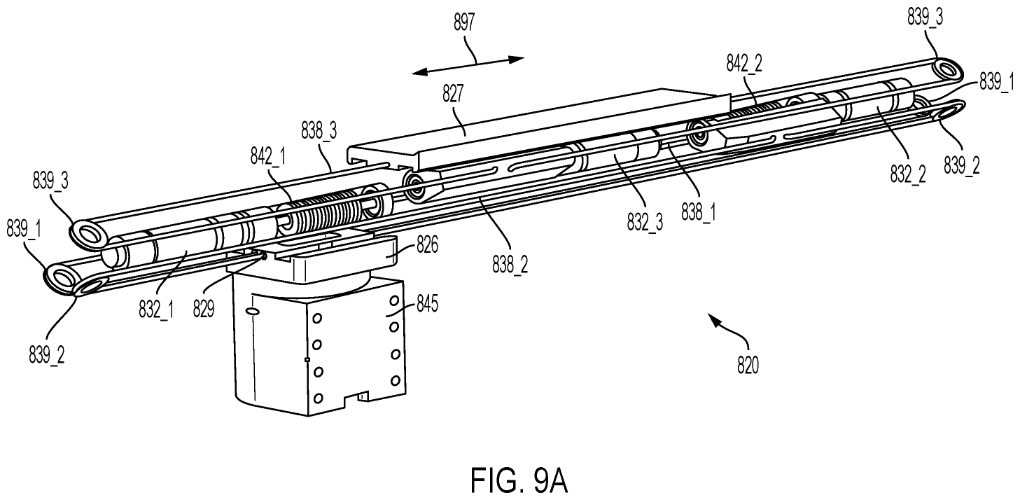

FIGS. 8-10 depict another embodiment of translation drive mechanisms and components thereof that can be used with a rail assembly of various embodiments, such as for example, with rail assemblies 120 or 220. FIG. 8A shows a rail assembly 820 mounted to a table assembly 801 and FIG. 8B shows a portion of a rail assembly 820 is shown in isolation, which can be used as rail assembly 120 or 220. Aspects of the table assembly and manipulators are not shown and not described here, but can be similar to the configurations and components described above with reference to other embodiments, such as table assemblies 101, 201, 301 and respective components. The rail assembly 820 includes a movable rail 821, two moveable manipulator carriages 826 carrying coupling portions 845 configured to mount and support manipulator arms (not shown) moveably mounted to the movable rail 821, and a rail mounting carriage 827 configured to mount the moveable rail 821 to the table assembly 801 (e.g., either to the platform assembly or support column as described above with reference to other embodiments). The rail 821 includes engagement features on opposing sides that are complementary to and engageable with engagement features on the carriages 826 and 827, similar to engagement features 122, 123, 222, 223, 125, 128, 228 described above, to allow the carriages to suspend from and ride along the rail 821.

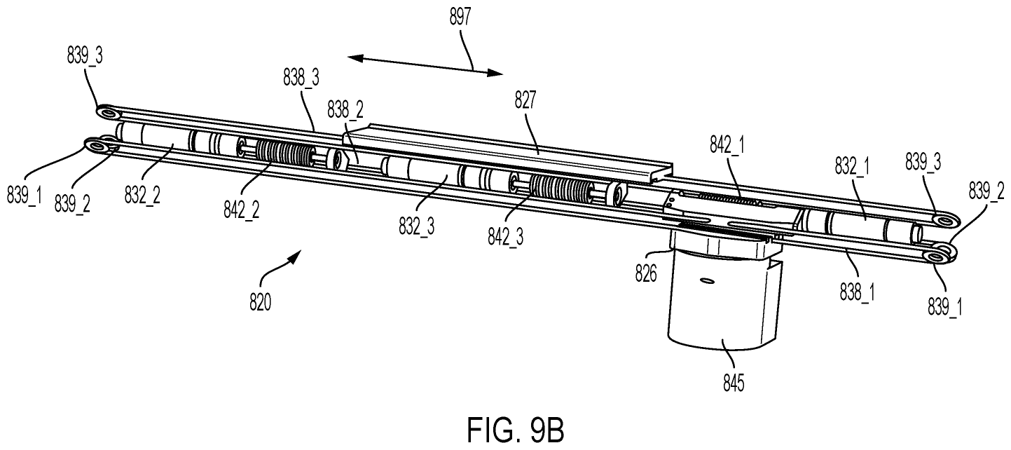

To drive translation of the moveable manipulator carriages 826 and rail 821 relative to each other, and the rail 821 and rail mounting carriage 827 relative to each other, translation drive mechanisms that include a cable, pulley, and capstan arrangement are used. FIGS. 9A and 9B are perspective views of FIG. 8B with the outer housing structure of the rail 821 removed to better illustrate the translation drive mechanism components, with the views of FIGS. 9A and 9B being taken from opposite sides. Three translation drive mechanisms are shown, two of which are respectively operably coupled to drive relative translation of the carriages 826 (only one of the carriages 826 shown in FIGS. 8A and 8B is illustrated in FIGS. 9A and 9B for simplicity) and rail 821, and one of which is operably coupled to drive relative translation of rail mounting carriage 827 and rail 821. More specifically, due to the fixed mounting of the rail mounting carriage 827 relative to the platform assembly (not shown), the rail 821 moves relative to the rail mounting carriage 827, and the moveable manipulator carriages 826 move relative to the rail 821.

Each translation drive mechanism includes a cable 838_1, 838_2, 838_3 looped around a pair of pulleys 839_1, 839_2, 839_3 disposed at opposite end portions of the rail 821 along the longitudinal dimension 897. The cables 838_1, 838_2, 838_3 further comprise opposite free end portions wrapped in opposite directions around a capstan 842_1, 842_2, 842_3 (third capstan 842_3 is not visible in FIG. 9A because hidden by housing) oriented with its axis of rotation generally along the longitudinal dimension 897 of the rail 821. An actuator 832_1, 832_2, 832_3 is operably coupled to each capstan 842_1, 842_2, 842_3 to drive rotation of the capstan 842_1, 842_2, 842_3. The manipulator carriages 826 (only one of which is illustrated) and rail mounting carriage 827 are respectively coupled in a fixed manner to a continuous length portion of the cables 838_1, 838_2, 838_3 that extend between the pulleys 839_1, 839_2, 839_3 opposite to where the capstan 842_1, 842_2, 842_3 is located between the pulleys 839_1, 839_2, 839_3.

Actuation of a capstan 842_1, 842_2, 842_3 by respective actuator 832_1, 832_2, 832_3 to rotate in a given direction causes one free end portion of the cable 838_1, 838_2, 838_3 to wrap further onto the capstan 842_1, 842_2, 842_3 and the other free end portion of the cable 838_1, 838_2, 838_3 to wrap off the capstan 842_1, 842_2, 842_3 (also referred to as paying in and paying out of the cable relative to the capstan). This in turn causes the continuous length portion of the cable between pulleys 839_1, 839_2, 839_3 to translate in a given direction, thereby causing translation in the same direction of the carriage 826, 827 attached to the continuous length portion of the cable 838_1, 838_2, 838_3. Rotation of the capstan 842_1, 842_2, 842_3 in the direction opposite of the given direction reverses which free end portion of the cable 838_1, 838_2, 838_3 is wrapped onto and off of the capstan 842_1, 842_2, 842_3 and so too the direction of translation of the continuous length portion of the cable 838_1, 838_2, 838_3 and corresponding attached carriage 826, 827.

The actuators 832 can be electric motors or other actuation devices (e.g., hydraulic, pneumatic, or other devices to provide a motive force) configured to drive rotation of a corresponding shaft to which the respective capstan can be coupled.

As depicted in FIGS. 9A, to conserve space for the three translation drive mechanisms housed in the rail 821, each of the manipulator carriages 826 (only one of which is shown in FIG. 9A, with two shown in FIGS. 8A and 8B) can be provided with a through-passage to allow the cable that a respective carriage 826 is not operably coupled to translate to pass freely through the carriage as it moves to drive the other carriage 826. For example, carriage 826 shown in FIG. 9A has a through-passage 829 through which cable 838_2 passes. Further, the actuator/capstan pairs for each drive mechanism can be respectively placed at opposite end portions of the rail 821 for the manipulator carriages 826 and at a middle portion of the rail mounting carriage 821 for the carriage 827. The cables 838_1, 838_2 and corresponding pulleys 839_1, 839_2 operably coupled to drive the manipulator carriages 826 can also be placed in a lower portion of the rail 821, while the cable 838_3 and pulleys 839_3 operably coupled to carriage 827 is placed above the cables 838_1, 838_2.

The actuators 832_1, 832_2, 832_3 and pulleys 839_1, 839_2, 839_3 can be mounted in a fixed position relative to the rail 821, and in some embodiment so are the capstans 842_1, 842_2, 842_3. In another embodiment, to further conserve space while maximizing the distance the continuous portion of a cable 838_1, 838_2, 838_3 can travel without the cable 838_1, 838_2, 838_3 unwrapping from the capstan 842_1, 842_2, 842_3, a traveling capstan arrangement can be used.

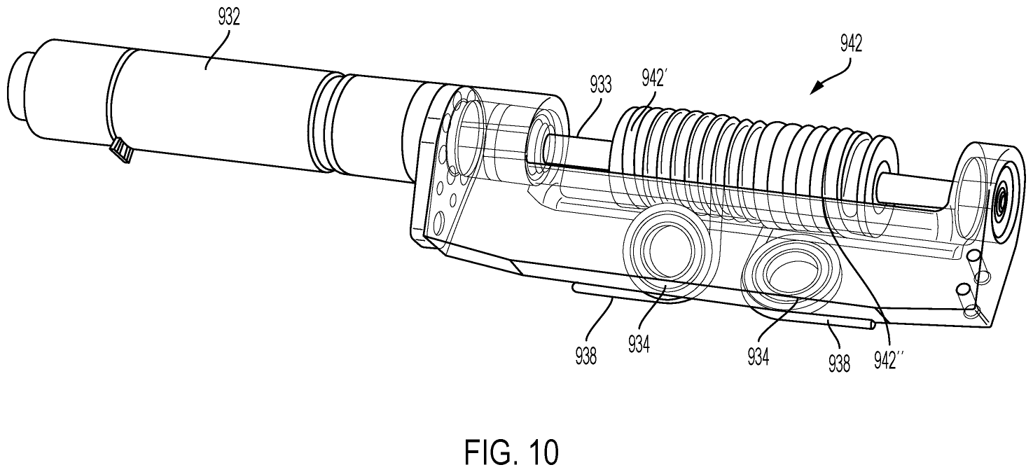

FIG. 10 illustrates a detailed view showing an embodiment of a traveling capstan arrangement that can be used as a nonlimiting embodiment of the capstans of the translation drive mechanisms of FIGS. 8 and 9. In the embodiment of FIG. 10, the actuator 932 includes a drive shaft in the form of a ball spline shaft 933. A capstan 942 is coupled to the ball spline shaft 933. The capstan 942 includes a first portion 942′ and a second portion 942″ having outer threaded surfaces. The respective opposite free end portions of a cable 938 are secured to and wrap in opposite directions on the respective first portion 942′ and second portion 942″ of the capstan 942. In this way, the portion of the cable 938 wrapped around first portion 942′ and the portion of cable 938 wrapped around second portion 942″ respectively wrap and unwrap from the capstan 942 oppositely in response to rotation of the capstan 942 in a given direction.

The balls spline shaft 933 coupling to the capstan 942 allows the capstan 942 to travel along the ball spline shaft 933 in differing directions depending on the direction of rotation of the ball spline shaft 933 as driven by actuator 932. As the capstan 942 moves long the ball spline shaft 933, the portions of the cable 938 can be paid in or out (wrap on or off) the respective portions 942′, 942″ oppositely depending on the direction of travel. This can provide a relatively compact arrangement of the capstan 942 to be used while still providing a relatively large range of translational motion of the continuous portion of the cable 938 (not shown in FIG. 10) and thus of the attached carriage.

To assist with guiding the cable 938 onto and off of the capstan 942 and routing the cable toward the pulleys associated with the cable 938 (such as pulleys 839 of FIGS. 8 and 9, with pulleys not shown 10 FIG. 10), a pair of redirecting pulleys 934 can be used. The redirecting pulleys 934 can be fixed in a housing (show transparent) to which the actuator 932, capstan 942, and ball spline shaft 933 are fixedly coupled. Openings in the housing can permit the cable 938 to exit the housing and be routed in opposite directions to the pair of pulleys the cable is coupled to.

The traveling capstan arrangement of FIG. 10 can in various embodiments have a configuration and operation like that disclosed in WO 2022/192109, published Sep. 15, 2022, the entire contents of which are incorporated by reference herein. This is just one nonlimiting embodiment of a traveling capstan arrangement that can be used, and other traveling capstan arrangements are contemplated as within the scope of the present disclosure. In another embodiment, as noted above, it is contemplated that the capstan arrangement could be fixed, and if redirecting pulleys are employed, the redirecting pulleys can be moveable to align the cable at a desired fleet angle with the capstan.

FIGS. 5A-5D show the platform assembly 210 and the rail assembly 220 in various configurations illustrative of a range of motion provided by the rail assembly 220 and various ranges of motion of the platform assembly 210 in some embodiments. Those having ordinary skill in the art will appreciate that the various configurations illustrated in FIGS. 5A-5D are non-limiting and other configurations are attainable in view of the various degrees of freedom of the rail 221, platform assembly sections 203, carriages 226, 227, and manipulators 240. In FIG. 5A, the rail mounting carriage 227 is positioned at a middle portion of the rail 221, such that the rail 221 is situated near the middle sections 203_2 and 203_3 of the platform assembly 210, with some portion of the length of the rail 221 extending near section 203_4. In FIG. 5A, the platform assembly 210 is in a neutral position with the manipulators 240 in a deployed state.

In FIG. 5B, the rail mounting carriage 227 (not visible in FIG. 5B) is positioned at a head end portion of the rail 221 (toward the left end of the rail 221 in the figure), such that the rail 221 has translated to the right from FIG. 5A and is situated near the foot end portion of the table assembly 220 (under the middle section 203_3 and the foot section 203_4). FIG. 5B also illustrates optional declined and inclined configurations the middle section 203_2 can be placed in in this positioning of the rail 221 (the declined configuration is illustrated in dashed lines and the inclined configuration in solid). The positioning of the rail assembly 220 in the configuration of FIG. 5B allows the first end section 203_1 to be declined or inclined (not illustrated) relative to the middle section 203_2, and/or allows the middle section 203_2 to be declined or inclined relative to the middle section 203_3 as illustrated in FIG. 5B, without interference (collision) with the rail assembly 220. Moreover, in this configuration of the rail assembly 220, the manipulators 240 can be moved in a longitudinal direction beyond a foot end of the platform assembly 210 (i.e., beyond the foot section 203_4) and then in a lateral direction such that the manipulators 240 are positioned along the laterally extending side of the platform assembly 210, as shown in FIG. 5B, instead of along the longitudinally extending side, as shown in FIG. 5A. This allows the manipulators to be positioned out of the way of operations performed along the longitudinally extending side of the platform assembly 210, such as an operation of transferring a patient from a gurney to the platform assembly 210.

In FIG. 5C, the rail mounting carriage 227 is positioned at a right end portion of the rail 221, such that the rail 221 is near a left end portion of the platform assembly 210. This configuration of the rail assembly 220 can allow the second end section 203_4 to be declined relative to the middle section 203_3 without collision with the rail assembly 220. FIG. 5C also illustrates the second end section 203_4 in declined and neutral configurations.

In FIG. 5D, inclined and declined configurations of the platform assembly 210 as a whole are illustrated. These configurations can be achieved by rotating the middle section 203_3 relative to the support column about an axis parallel to the lateral dimension 299. As shown in FIG. 5D, the rail assembly 220 and the manipulators 240 move along with the platform assembly 210 in these embodiments because the rail assembly 220 is attached to the platform assembly 210. In other embodiments (not illustrated), the rail assembly 220 can be attached to the support column 202.

Thus, the rail assembly 220 provides a wide range of motion for the manipulators 240, which can allow the manipulators 240 to be positioned nearly anywhere along the longitudinal dimension 298 of the platform assembly 210, including positioning the manipulators 240 beyond or outside of a head or foot end of the platform assembly such that the manipulators 240 can be clear from the entire longitudinal dimension 298 of the platform assembly. Moreover, not only does the rail assembly 220 provide a wide range of motion for the manipulators 240, the rail assembly 220 does so while also minimizing interference between the rail assembly 220 and the platform assembly 210, personnel, and other equipment. In particular, the rail assembly is movable between different positions as noted above based on the configuration of the platform assembly 210 and/or based on the needs of a particular operation such that the rail 221 is positioned out of the way of the portions of the platform assembly 210, personnel, or other equipment as needed.

Furthermore, the rail assembly 220 is configured to remain within an envelope 208 defined by the platform assembly 210 throughout all motion of the rail 221 relative to the platform assembly 210 and/or support column 202. This further reduces the degree of interference between the rail assembly 220 and other equipment, as will be described in greater detail below. The envelope 208 (see FIG. 3) is defined by the platform assembly 210 in a similar manner as the envelope 108, and thus duplicative description thereof is omitted.

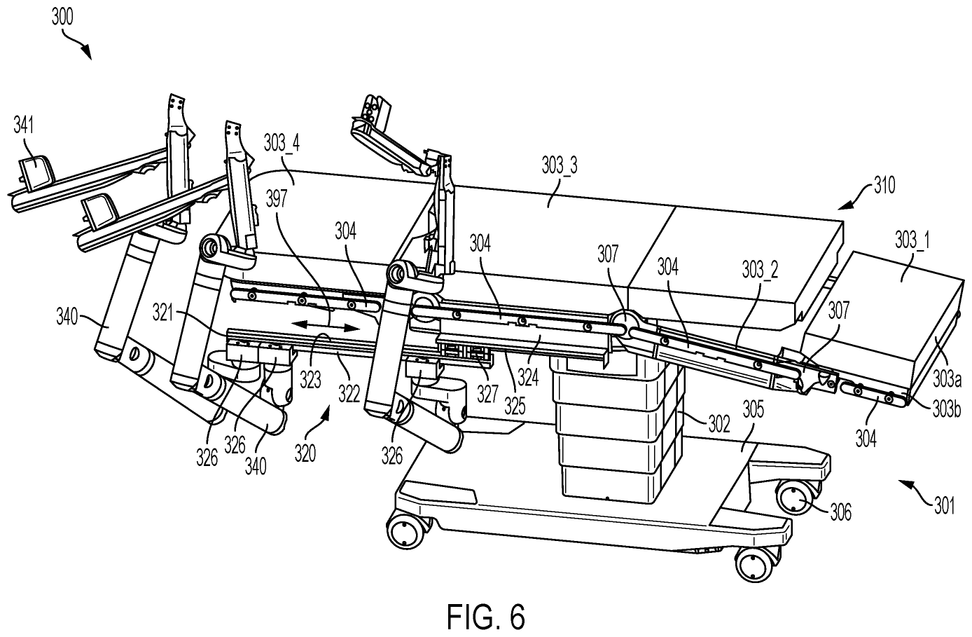

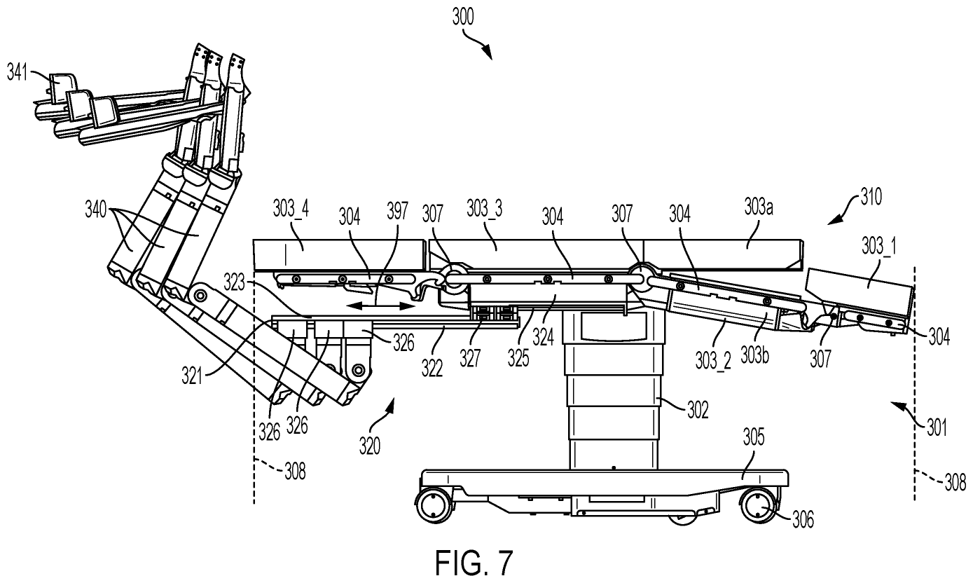

Turning now to FIGS. 6 and 7, an embodiment of a table-mounted manipulator system 300 (“system 300”) is described below. The system 300 can be used as the system 100, and some components of the system 300 can be used as components of the system 100 described above. Thus the descriptions of the components of the system 100 above are applicable to the related components of the system 300, and duplicative descriptions of these components are omitted below. The related components of the systems 100 and 300 are given reference numbers having the same right-most two digits—for example, 105 and 305. Although the system 300 is one embodiment of the system 100, the system 100 is not limited to the system 300.

As shown in FIGS. 3-5D, the system 300 includes a table assembly 301, a rail assembly 320 coupled to the table assembly, and multiple manipulators 340 coupled to the rail assembly 320. Each manipulator 340 carries one more instruments (not illustrated), which can be removably or permanently mounted thereon. The system 300 also can include a control system (not illustrated), a user input and feedback system (not illustrated), and/or an auxiliary system (not illustrated) similar to those described above in relation to the system 100. In some embodiments, the system 300 is configured as a computer-assisted, teleoperable medical system. In other embodiments, the system 300 is configured as a teleoperable system for use in non-medical contexts.

As shown in FIGS. 6 and 7, the table assembly 301 includes a platform assembly 310 configured to support the patient or inanimate workpiece, a support column 302 coupled to and supporting the platform assembly 310, and a base 305 coupled to the support column 302. The base 305 can be configured to contact the ground or other surface upon which the table assembly 301 rests, and in some embodiments the base 305 includes wheels 306 to allow movement of the system 300 along the ground or other surface. In some embodiments the support column 302 includes a telescoping support that can raise or lower the platform assembly 310 relative to the ground or other surface.

The platform assembly 310 includes multiple platform sections 303 configured to support the patient or workpiece. In particular, in the embodiment illustrated in FIGS. 6 and 7 the platform assembly 310 includes first end section 303_1, middle sections 303_2 and 303_3, and second end section 303_4, which are arranged in series and movably coupled together via joints 307, similar to the platform assembly 210 described above. The joints 307 allow adjacent platform sections 303 to pivot relative to one another as described above in relation to platform assembly 210. In some embodiments, some of the joints 307 can also allow for other motion between adjacent platform sections 303, such as relative translation along the longitudinal dimension 398 or relative rotation around a vertical axis. In some embodiments, the platform sections 303 include relatively rigid support portions 303b and softer cushion portions 303a attached to the support portions 303b, with a surface of the cushion portions 303a (i.e., the top surface in the orientation illustrated in FIG. 7) forming a support surface that contacts the patient or workpiece. In some embodiments, multiple platform sections 303 can share some components. For example, as illustrated in FIGS. 6 and 7, the middle platform sections 303_2 and 303_3 can share the same cushion portion 303a that extends across both platform sections 303_2 and 303_3.

In addition to moving individual platform section 303 relative to adjoining platform sections 303, the platform assembly 310 as a whole is movable relative to the support column 302. In some embodiments, the middle section 303_3 is coupled to the support column 302 by one or more joints (not illustrated), providing for motion between the middle section 303_3 and the column 302. In some embodiments, the motions of the platform assembly 310 can be similar to those of the platform assembly 210 described above, and thus duplicative description thereof is omitted.

As shown in FIGS. 6 and 7, the platform assembly 310 also optionally includes a number of accessory rails 304 attached to side surfaces of the support portions 303b of the platform sections 303.

As noted above, the system 300 includes multiple manipulators 340. In the embodiment illustrated in FIGS. 6 and 7, three manipulators 340 are present. In other embodiments, more or fewer manipulators 340 can be used. In the embodiment of FIGS. 6 and 7 all three manipulators 340 are positioned on the same longitudinally extending side of the platform assembly 310 and are coupled to the same rail assembly 320. In other embodiments (not illustrated) of the system 300, multiple rail assemblies 320 can be included (e.g., one on each longitudinally extending side of the platform assembly 310, with a first rail assembly 320 on a first longitudinally extending side of the platform assembly 310 and a second rail assembly 320 on a second longitudinally extending side of the platform assembly 310, wherein the second longitudinally extending side is opposite to the first longitudinally extending side). When multiple rail assemblies 320 are included, each rail assembly 320 can have one or more manipulators 340 coupled thereto (e.g., one, two, three, or more manipulators 340 can be coupled to each rail assembly 320). The manipulators 340 include a number of links and joints, as well as an instrument manipulator mount 341 configured to receive an instrument removably mounted thereon and including an interface with output couplers to transfer driving force or other inputs to the instrument.

As shown in FIGS. 6 and 7, the manipulators 340 are coupled to the table assembly 301 via the rail assembly 320. As shown in FIGS. 6 and 7, the rail assembly 320 includes a movable rail 321 (“rail 321”), movable manipulator carriages 326 coupled to the rail 321 and to the manipulators 340 (at least one movable manipulator carriage 326 per manipulator 340), and one or more rail mounting carriages 327 coupled to the rail 321 and the table assembly 301.

Each movable manipulator carriage 326 couples a respectively corresponding one of the manipulators 340 to the rail 321 such that the manipulators 340 can translate relative to the rail 321 along a longitudinal dimension 397 of the rail 321. In addition, each rail mounting carriage 327 couples the rail assembly 320 to the platform assembly 310 (e.g., to the middle section 303_3) such that the rail 321 can translate relative to the platform assembly 310 and the support column 302 along the longitudinal dimension 397 of the rail 321. In the embodiment of FIGS. 6 and 7, because the rail assembly 320 is coupled to the platform section 303_3, the longitudinal dimension 397 of the rail 321 is parallel to the longitudinal dimension 398 of the platform assembly 310 regardless of how the platform assembly 310 is moved relative to the support column 302.

As shown in FIGS. 6 and 7, the movable rail 321 includes first and second sets of engagement features 322 and 323 configured to engage with complementary engagement features of the carriages 326 and 327, respectively, to movably couple the rail 321 to the carriages 326 and 327. The first and second sets of engagement features 322 and 323 each includes tracks including flanges and/or grooves extending along the longitudinal dimension 397, and the complementary engagement features of the carriages 326 and 327 are configured to engage and ride along the flanges and/or in the grooves of the first and second sets of engagement features 322 and 323, respectively. In some embodiments, the complementary engagement features of the carriages 326 and 327 can include bearing devices (not illustrated) configured to reduce friction to facilitate easier translation, such as wheels, balls, plain bearing surfaces, etc.

In some embodiments, the carriages 326 and 327 are manually movable relative to the rail 321. In other embodiments, the carriages 326 and 327 include actuators (not illustrated) to drive motion of the carriages 326 and 327 relative to the rail 321. In some embodiments, the actuators can be housed within the carriages 326 and 327, unlike the rail assemblies 220, 820 in which the actuators were housed within the rail 221. The actuators can, for example, include motors configured to drive rotation of gears and/or wheels engaged with the engagement features 322 and 323 to cause translation of the carriages 326 and 327 relative to the rail 321.

In some embodiments, the rail mounting carriages 327 are coupled to the middle section 303_3 via a fixed rail 324 attached to the middle section 303_3, as shown in FIGS. 6 and 7. For example, in some embodiments a first end of the carriage 327 is movably coupled to the fixed rail 324, while a second end of the carriage 327 is movably coupled to the movable rail 321 as described above. This allows the rail mounting carriage 327 to not only move relative to the rail 321, but also to move relative to the platform assembly 310. Thus, in these embodiments, translation of the rail 321 relative to the table assembly 301 can occur through either one of (or both of) relative translation between the carriages 327 and the table assembly 301 (i.e., relative to the fixed rail 324) and relative translation between the carriages 327 and rail 321. The movability of the rail mounting carriages 327 relative to the table assembly 301 can allow for a greater range of motion of the manipulators 340 for a given size of rail 321 and/or a smaller rail 321 to be used for a given range of motion.

The rail assembly 320 and the platform assembly 310 can be movable through ranges of motion similar to those already described above in relation to the system 200, and thus duplicative description thereof is omitted. Thus, the rail assembly 320 provides a wide range of motion for the manipulators 340, which can allow the manipulators 340, where they attach to the carriages 326, to be positioned nearly anywhere along the length of the platform assembly 310. Moreover, not only does the rail assembly 320 provide a wide range of motion for the manipulators 340, it does so while also minimizing interference between the rail assembly 320 and the platform assembly 310, personnel, and other equipment. In particular, the rail assembly is movable between different positions as noted above based on the configuration of the platform assembly 310 and/or based on the needs of a particular operation such that the rail 321 is positioned out of the way of the portions of the platform assembly 310, personnel, or other equipment as needed.

Furthermore, the rail assembly 320 is configured to remain within an envelope 308 defined by the platform assembly 310 throughout all motion of the rail 321 relative to the platform assembly 310 and/or support column 302. This further reduces the degree of interference between the rail assembly 320 and other equipment. The envelope 308 (see FIG. 7) is defined by the platform assembly 310 in a similar manner as the envelope 108, and thus duplicative description thereof is omitted.

The embodiments described herein may be well suited for use in any of a variety of medical procedures, as described above. Such procedures could be performed, for example, on human patients, animal patients, human cadavers, animal cadavers, and portions or human or animal anatomy. Medical procedures as contemplated herein include any of those described herein and include, for non-surgical diagnosis, cosmetic procedures, imaging of human or animal anatomy, gathering data from human or animal anatomy, training medical or non-medical personnel, and procedures on tissue removed from human or animal anatomies (without return to the human or animal anatomy). Even if suitable for use in such medical procedures, the embodiments can also be used for benchtop procedures on non-living material and forms that are not part of a human or animal anatomy. Moreover, some embodiments are also suitable for use in non-medical applications, such as industrial robotic uses, and sensing, inspecting, and/or manipulating non-tissue work pieces. In non-limiting embodiments, the techniques, methods, and devices described herein can be used in, or be part of, a computer-assisted surgical system employing robotic technology such as the da Vinci® Surgical Systems commercialized by Intuitive Surgical, Inc., of Sunnyvale, California. Those skilled in the art will understand, however, that aspects disclosed herein can be embodied and implemented in various ways and systems, including manually operated instruments and computer-assisted, teleoperated systems, in both medical and non-medical applications. Reference to the daVinci® Surgical Systems are illustrative and not to be considered as limiting the scope of the disclosure herein.

As used herein and in the claims, terms such as computer-assisted manipulator system, teleoperable manipulator system, or the like should be understood to refer broadly to any system including one or more controllable kinematic structures (“manipulators”) that are movable and controllable at least in part through the aid of an electronic controller (with or without human inputs). Such systems are occasionally referred to in the art and in common usage as robotically assisted systems or robotic systems. Such systems include systems that are controlled by a user (for example through teleoperation), by a computer automatically (so-called autonomous control), or by some combination of these. In examples in which a user controls at least some of the operations of the manipulator, an electronic controller (e.g., a computer) can facilitate or assist in the operation. The term “computer” as used in “computer-assisted manipulator systems” refers broadly to any electronic control device for controlling, or assisting a user in controlling, operations of the manipulator, and is not intended to be limited to things formally defined as or colloquially referred to as “computers.” For example, the electronic control device in a computer-assisted manipulator system could range from a traditional “computer” (e.g., a general-purpose processor plus memory storing instructions for the processor to execute) to a low-level dedicated hardware device (analog or digital) such as a discrete logic circuit or application specific integrated circuit (ASIC), or anything in between. Further, manipulator systems can be implemented in a variety of contexts to perform a variety of procedures, both medical and non-medical. Thus, although some examples described in greater detail herein are focused on a medical context, the devices and principles described herein are also applicable to other contexts, such as industrial manipulator systems.

It is to be understood that both the general description and the detailed description provide example embodiments that are explanatory in nature and are intended to provide an understanding of the present disclosure without limiting the scope of the present disclosure. Various mechanical, compositional, structural, electrical, and operational changes can be made without departing from the spirit and scope of this description and the claims. In some instances, well-known circuits, structures, and techniques have not been shown or described in detail in order not to obscure the embodiments. Like numbers in two or more figures represent the same or similar elements.

Further, the terminology used herein to describe aspects of the invention, such as spatial and relational terms, is chosen to aid the reader in understanding example embodiments of the invention but is not intended to limit the invention. For example, spatial terms—such as “beneath”, “below”, “lower”, “above”, “upper”, “proximal”, “distal”, “up”, “down”, and the like—are used herein to describe directions or one element's or feature's spatial relationship to another element or feature as illustrated in the figures. These spatial terms are used relative to the figures and are not limited to a particular reference frame in the real world. Thus, for example, the direction “up” in the figures does not necessarily have to correspond to an “up” in a world reference frame (e.g., away from the Earth's surface). Furthermore, if a different reference frame is considered than the one illustrated in the figures, then the spatial terms used herein may need to be interpreted differently in that different reference frame. For example, the direction referred to as “up” in relation to one of the figures may correspond to a direction that is called “down” in relation to a different reference frame that is rotated 180 degrees from the figure's reference frame. As another example, if a device is turned over 180 degrees in a world reference frame as compared to how it was illustrated in the figures, then an item described herein as being “above” or “over” a second item in relation to the Figures would be “below” or “beneath” the second item in relation to the world reference frame. Thus, the same spatial relationship or direction can be described using different spatial terms depending on which reference frame is being considered. Moreover, the poses of items illustrated in the figure are chosen for convenience of illustration and description, but in an implementation in practice the items can be posed differently.

In addition, the singular forms “a”, “an”, and “the” are intended to include the plural forms as well, unless the context indicates otherwise. And, the terms “comprises”, “comprising”, “includes”, and the like specify the presence of stated features, steps, operations, elements, and/or components but do not preclude the presence or addition of one or more other features, steps, operations, elements, components, and/or groups. Components described as coupled can be electrically or mechanically directly coupled, or they can be indirectly coupled via one or more intermediate components, unless specifically noted otherwise. Mathematical and geometric terms are not necessarily intended to be used in accordance with their strict definitions unless the context of the description indicates otherwise, because a person having ordinary skill in the art would understand that, for example, a substantially similar element that functions in a substantially similar way could easily fall within the scope of a descriptive term even though the term also has a strict definition.

Elements and their associated aspects that are described in detail with reference to one embodiment can be, whenever practical, included in other embodiments in which they are not specifically shown or described. For example, if an element is described in detail with reference to one embodiment and is not described with reference to a second embodiment, the element may nevertheless be claimed as included in the second embodiment.

As used herein, “proximal” and “distal” are spatial/directional terms that describe locations or directions based on their relationship to the two ends of a kinematic chain. “Proximal” is associated with the end of the kinematic chain that is closer to the base or support of the chain, while “distal” is associated with the opposite end of the kinematic chain, which often includes an end effector of an instrument. When used in to refer to locations or to portions of a component, proximal and distal indicate the relative positions of the locations or portions relative to the base of the chain, with the proximal location or portion being closer to the base (closer here referring to proximity along the kinematic chain, rather than absolute distance). When used to refer to a direction, “proximal” refers to directions that point generally from a given location along a kinematic chain towards a more proximal location along the kinematic chain, and “distal” refers to directions that point from the given location towards a more distal location along the kinematic chain.

Unless otherwise noted herein or implied by the context, when terms of approximation such as “substantially,” “approximately,” “about,” “around,” “roughly,” and the like, are used in conjunction with a stated numerical value, property, or relationship, such as an end-point of a range or geometric properties/relationships (e.g., parallel, perpendicular, straight, etc.), this should be understood as meaning that mathematical exactitude is not required for the value, property, or relationship, and that instead a range of variation is being referred to that includes but is not strictly limited to the stated value, property, or relationship. In particular, the range of variation around the stated value, property, or relationship includes at least: any inconsequential variations; those variations that are typical in the relevant art for the type of item in question due to manufacturing or other tolerances; and/or variations that are within ±5% of the stated value, property, or relationship unless indicated otherwise.

As used herein, “transverse” refers to a positional relationship of two items in which one item is oriented crosswise at an angle relative to the other item, such as being substantially or generally perpendicular to the other item. As used herein, “transverse” includes, but does not require, an exactly perpendicular relationship.

Further modifications and alternative embodiments will be apparent to those of ordinary skill in the art in view of the disclosure herein. For example, the devices and methods can include additional components or steps that were omitted from the diagrams and description for clarity of operation. Accordingly, this description is to be construed as illustrative only and is for the purpose of teaching those skilled in the art the general manner of carrying out the present teachings. It is to be understood that the various embodiments shown and described herein are to be taken as exemplary. Elements and materials, and arrangements of those elements and materials, can be substituted for those illustrated and described herein, parts and processes can be reversed, and certain features of the present teachings can be utilized independently, all as would be apparent to one skilled in the art after having the benefit of the description herein. Changes can be made in the elements described herein without departing from the spirit and scope of the present teachings and following claims.

It is to be understood that the particular examples and embodiments set forth herein are non-limiting, and modifications to structure, dimensions, materials, and methodologies can be made without departing from the scope of the present teachings.

Other embodiments in accordance with the present disclosure will be apparent to those skilled in the art from consideration of the specification and practice of the invention disclosed herein. It is intended that the specification and examples be considered as exemplary only, with the following claims being entitled to their fullest breadth, including equivalents, under the applicable law.

1. A teleoperable manipulator system, comprising:
a table assembly comprising:
a support column; and
a platform assembly coupled to the support column and configured to support a body, wherein the platform assembly comprises lateral and longitudinal dimensions defining outer lateral and longitudinal boundaries of the platform assembly;
a rail coupled to the table assembly, the rail being translatable relative to the support column along a longitudinal dimension of the rail; and
one or more manipulators coupled to the rail and translatable relative to the rail along the longitudinal dimension of the rail,
wherein the rail is configured to remain within at least the outer lateral boundaries of the platform assembly throughout all motion of the rail relative to the support column.

2. The system of claim 1,
wherein the rail is configured to remain within the outer longitudinal boundaries of the platform assembly throughout all motion of the rail relative to the support column.

3. The system of claim 1,
wherein the platform assembly comprises a plurality of sections movable relative to one another to change a configuration of the platform assembly between a plurality of configurations, and
wherein the rail is movable between a plurality of positions to allow the platform assembly to move between the plurality of configurations without interference from the rail.

4. The system of claim 3,
wherein the plurality of sections comprise a first end section, one or more middle sections, and a second end section consecutively positioned along the longitudinal dimension of the platform assembly, and
wherein each of the first and second end sections are independently pivotable relative to the one or more middle sections.

5. The system of claim 3, further comprising:
one or more first carriages, each first carriage movably coupling one of the manipulators to the rail, each first carriage being drivable to cause translation of the manipulator coupled thereto relative to the rail along the longitudinal dimension of the rail; and
one or more second carriages movably coupling the rail to the table assembly, each second carriage being drivable to cause translation of the rail relative to the platform assembly and the support column.

6. The system of claim 5,
a control system communicably connected to the first and second carriages, the control system being configured to drive the first and second carriages to position the rail and the manipulators.

7. The system of claim 3,
wherein the plurality of sections comprise a first section and a second section positioned adjacent to and pivotable relative to the first section,
wherein in a first state the second section is declined relative to the first section and at least a portion of the second section occupies a given region of space,
wherein the rail is translatable relative to the support column between a first position and a second position, in the first position a portion of the rail occupies the given region of space, in the second position the rail does not occupy the given region of space.

8. The system of claim 7, further comprising:
a control system configured to, in response to determining a transition to the first state is going to occur, cause the rail to move from the first position to the second position.

9. The system of claim 1, further comprising:
one or more carriages, each carriage movably coupling one of the manipulators to the rail, each carriage being drivable in translation along the longitudinal dimension of the rail so as to cause translation of the manipulator coupled thereto relative to the rail along the longitudinal dimension of the rail.

10. (canceled)

11. The system of claim 9, further comprising a drive mechanism operably coupled to drive translation of the one or more carriages along the longitudinal dimension of the rail, wherein the drive mechanism comprises:
a lead screw;
one or more follower nuts engaged with the lead screw and coupled with respectively corresponding ones of the one or more carriages; and
one or more actuators operably coupled to the follower nuts and configured to rotate the one or more follower nuts relative to the lead screw.

12. The system of claim 9, further comprising a drive mechanism operably coupled to drive translation of the one or more carriages along the longitudinal dimension of the rail, wherein the drive mechanism comprises:
one or more cables coupled with respectively corresponding ones of the one or more carriages;
one or more capstans, wherein the one or more cables are at least partially wrapped around the one or more capstans, respectively; and
one or more actuators respectively operably coupled to the one or more capstans and configured to rotate the one or more capstans,
wherein the one or more cables are configured to be moved in translation in response to rotation of the one or more capstans.

13. The system of claim 12,
wherein the one or more carriages comprise a first carriage and a second carriage and the system further comprises a rail mounting carriage movably coupling the rail to the platform assembly,
wherein the one or more cables comprise a first cable coupled with the first carriage, a second cable coupled with the second carriage, and a third cable coupled with the rail mounting carriage,
wherein the one or more capstans comprises a first capstan, a second capstan, and a third capstan,
wherein the first cable is at least partially wrapped around the first capstan, the second cable is at least partially wrapped around the second capstan.

14. The system of claim 11, further comprising:
one or more rail mounting carriages movably coupling the rail to the platform assembly, the one or more rail mounting carriages being drivable to cause translation of the rail relative to the platform assembly and the support column.

15. The system of claim 14, further comprising:
a second lead screw;
one or more second follower nuts engaged with the second lead screw and coupled with respectively corresponding ones of the one or more rail mounting carriages; and
one or more second actuators operably coupled to the second follower nuts to drive translation of respectively corresponding ones of the one or more rail mounting carriages relative to the rail by driving rotation of the respectively corresponding second follower nuts relative to the second lead screw.

16. The system of claim 14, further comprising:
a fixed rail coupled to the platform assembly or to the support column,
wherein the one or more rail mounting carriages are movably coupled to the fixed rail and are translatable relative to the platform assembly along the fixed rail.

17. The system of claim 1,
wherein the platform assembly is tiltable relative to the support column; and
the rail is configured to tilt along with the platform assembly relative to the support column.

18. The system of claim 17,
wherein the platform assembly comprises a plurality of sections movable relative to one another to change a configuration of the platform assembly between a plurality of configurations,
wherein a first section of the plurality of sections is coupled to the support column and is tiltable relative to the support column, and
wherein the rail is coupled to the first section.

19. The system of claim 1, further comprising:
an accessory rail coupled to a longitudinal side of the platform assembly, the accessory rail configured to mount auxiliary medical equipment,
wherein the outer lateral boundaries of the platform assembly are defined by outermost edges of the platform assembly along the longitudinal side excluding the accessory rail.

20. The system of claim 1, further comprising:
a rail mounting carriage coupled to the platform assembly or to the support column,
wherein the rail is movably coupled to the rail mounting carriage and is configured to move along the rail mounting carriage to cause translation of the rail relative to the support column along the longitudinal dimension of the rail; and
a drive mechanism operably coupled to drive movement of the rail along the rail mounting carriage, wherein the drive mechanism comprises a lead screw drive mechanism or a capstan drive mechanism.

21.-23. (canceled)

24. A rail assembly for mounting manipulators to a table assembly, the rail assembly comprising:
a rail having a longitudinal dimension;
a rail mounting carriage movably engaged with the rail and configured to couple the rail to a table assembly;
first and second manipulator mounting carriages movably engaged with the rail at an opposite side of the rail as the rail mounting carriage; and
a drive mechanism housed in an interior of the rail, the drive mechanism comprising:
a first capstan assembly,
a first cable operably coupled to the first capstan assembly and to the first manipulator carriage, the first cable passing through the second manipulator carriage
a second capstan assembly,
a second cable operably coupled to the second capstan assembly and the second manipulator carriage, the second cable passing through the first manipulator carriage
a third capstan assembly,
a third cable operably coupled to the third capstan assembly and the rail mounting carriage,
wherein the first, second, and third capstan assemblies are arranged in a series along the longitudinal dimension of the rail.
